(19) United States
(12) Patent Application Publication
(10) Pub. No.: US 2011/0223633 A1
(43) Pub. Date: Sep. 15, 2011

(54) METHODS AND REAGENTS FOR ENRICHMENT AND CHARACTERIZATION OF PHOSPHORYLATED BIOMOLECULES

(76) Inventors: Alexey V. Pshezhetsky, Mont-Royal (CA); Ilya E. Nifant'Ev, Moscow (RU)

(21) Appl. No.: 13/125,267

(22) PCT Filed: Oct. 20, 2009
(86) PCT No.: PCT/CA09/01458

§ 371 Date: Apr. 20, 2011

Related U.S. Application Data
(60) Provisional application No. 61/107,103, filed on Oct. 21, 2008.

(51) Int. Cl. C12P 21/06; C08B 37/02; C08B 37/00

(52) U.S. Cl. 435/68.1; 536/112; 536/121

(57) ABSTRACT

An affinity matrix comprising a metal ion covalently attached thereto and methods for making and using the same are described. The matrix has affinity for various phosphorylated biomolecules, such as phosphoproteins/phosphopeptides. The matrix may be used in a variety of different applications, including phospho-biomolecule (e.g., phosphoprotein and phosphopeptides) enrichment/purification and characterization applications. Also provided are kits and systems that include the matrix.

### CROSS REFERENCE TO RELATED APPLICATIONS

This application claims the benefit, under 35 U.S.C. §119(e), of U.S. provisional application Ser. No. 61/107,103, filed on Oct. 21, 2008, which is incorporated herein by reference in its entirety.

### TECHNICAL FIELD

The present invention generally relates to affinity purification/enrichment, and more particularly to reagents and methods for purification/enrichment of biomolecules such as phosphorylated biomolecules.

### BACKGROUND ART

Phosphorylation is the most frequent and important post-translational modification of proteins. Phosphorylation regulates protein folding, function (e.g., enzymatic activity), cellular localization, complex formation and stability, and, consequently plays an important role in signal transduction, metabolism, and cell division. Over 50% of all proteins are thought to be phosphorylated and over 100,000 estimated phosphorylation sites are predicted in the human proteome (Kalume D E, et al., Curr Opin Chem Biol. 2003 7(1): 64-9). The direct identification of phosphopeptides by mass spectroscopy (MS) is difficult because of multiple suppression effects. The majority of current phosphoproteomic methods rely therefore on the selective purification of phosphorylated peptides or proteins. Various approaches have been described for enrichment of phosphoproteins, such as using antibodies specific to phosphorylated amino acid residues, by chemical modifications of the phosphate groups, using immobilized metal affinity chromatography (IMAC) or metal-oxide chromatography, but these approaches are associated with significant drawbacks including poor yields and/or low specificity. Therefore, despite intensive research dedicated to development of phosphoproteomic methods, the identification and quantification of phosphoproteins present in small quantities still remains a challenging task, highlighting the need for development of novel technologies.

The present description refers to a number of documents, the content of which is herein incorporated by reference in their entirety.

### SUMMARY OF THE INVENTION

The present invention relates to reagents and methods for the enrichment and characterization of phosphorylated biomolecules, such as phosphorylated polypeptides.

In a first aspect, the present invention provides an affinity matrix comprising a solid support and a metal ion covalently attached thereto.

In another aspect, the present invention provides a method for preparing an affinity matrix comprising a solid support and a metal ion covalently attached thereto, said method comprising:
(a) contacting a solid support comprising hydroxyl groups attached thereto with an organometallic compound soluble in an organic solvent; and
(b) contacting the solid support of (a) with a source of free hydroxyl groups, thereby obtaining an affinity matrix comprising a solid support and a metal ion covalently attached thereto.

In another aspect, the present invention provides a method for obtaining a sample enriched in phosphorylated peptides from a protein-comprising sample, said method comprising:
(a) contacting the protein-comprising sample with matrix comprising a metal ion having a 3⁺ oxidation state;
(b) eluting the proteins from said matrix to obtain a phosphoprotein enriched sample;
(c) incubating the phosphoprotein enriched sample of (b) with a proteolytic enzyme thereby to obtain peptides;
(d) contacting the peptides of (c) with a matrix comprising a metal ion having a 3⁺ oxidation state; and
(e) eluting the peptides from the matrix of (d);
   thereby obtaining a sample enriched in phosphorylated peptides.

In another aspect, the present invention provides a method for analyzing the phosphopeptide content of a sample comprising (i) separating the phosphopeptides comprised in the sample enriched in phosphorylated peptides obtained by the above-mentioned method by two-dimensional liquid chromatography, and (ii) identifying the separated peptides by mass spectrometry.

In another aspect, the present invention provides a kit comprising: (a) a solid support comprising hydroxyl groups attached thereto and (b) an organometallic compound.

In another aspect, the present invention provides a method for determining the ratio of phosphorylated to non-phosphorylated forms of a polypeptide comprising:

(a) contacting a sample comprising said polypeptide with a proteolytic enzyme thereby to obtain peptides;

(b) labelling a first fraction of the peptides of (a) with a first tag (e.g., a first isotopic tag) and a second fraction of the peptides of (a) with a second tag (e.g., a second isotopic tag), wherein said first and second tags are different (e.g., said first and second isotopic tags are of different masses);

(c) contacting said first or second fraction with a dephosphorylating agent;

(d) submitting a comparable amount of said first and second fraction to a detection method capable of distinguishing between said first and second tags (e.g., mass spectroscopy) to obtain a detection profile (e.g., a mass spectroscopy profile); and

(e) determining the ratio of phosphorylated to non-phosphorylated forms of said polypeptide in accordance with the detection (e.g., mass spectroscopy) profile obtained.

In another aspect, the present invention provides a method for enriching phosphorylated biomolecules from a sample, said method comprising:

(a) contacting the sample with the above-mentioned matrix;

(b) eluting the phosphorylated biomolecules from said matrix to obtain a phosphorylated biomolecule-enriched sample.

In an embodiment, the above-mentioned metal ion has a 2⁺ or 3⁺ oxidation state. In a further embodiment, the above-mentioned metal ion has a 3⁺ oxidation state.

In another embodiment, the above-mentioned affinity matrix is a phospho-affinity matrix.

In an embodiment, the above-mentioned metal ion is aluminum, (ii) gallium, or (iii) a combination of (i) and (ii).

In another embodiment, the above-mentioned solid support is a cross-linked polysaccharide. In a further embodiment, the above-mentioned polysaccharide is dextran. In another embodiment, the above-mentioned solid support is Sephadex™, in a further embodiment Sephadex™ G25.

In an embodiment, the above-mentioned polysaccharide comprises a monomeric moiety of formula I:

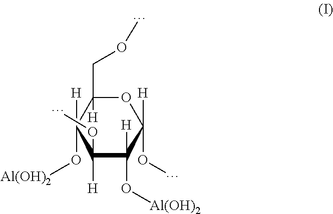

In an embodiment, the above-mentioned organic solvent is a non-polar solvent.

In an embodiment, the above-mentioned organometallic compound is an alkylate of a metal. In a further embodiment, the above-mentioned alkylate of a metal is a trialkyl aluminum, a trialkyl gallium, or a combination thereof. In a further embodiment, the above-mentioned trialkyl aluminum is triethylalkyl aluminum (Al₂(Et)₆). In another embodiment, the above-mentioned trialky gallium is triethylalkyl gallium (Ga₂(Et)₆).

In an embodiment, the above-mentioned organic solvent is hexane.

In another embodiment, the above-mentioned source of free hydroxyl groups is water.

In an embodiment, the above-mentioned method for preparing an affinity matrix further comprises (c) washing said solid support with an organic solvent. In a further embodiment, the above-mentioned organic solvent for washing said solid support is diethyl ether.

In a further embodiment, the above-mentioned method for preparing an affinity matrix further comprises (d) drying the matrix. In a further embodiment, the above-mentioned drying is performed under vacuum.

In an embodiment, the above-mentioned protein-comprising sample is a cell or tissue extract, and, wherein said method for obtaining a sample enriched in phosphorylated peptides further comprises before (a): incubating said cell or tissue extract with a buffer comprising a denaturing agent.

In an embodiment, the above-mentioned matrix is an iminodiacetic acid-based resin having aluminum ions bound thereto. In a further embodiment, the above-mentioned iminodiacetic acid-based resin is Sepharose™.

In another embodiment, the matrix is the above-mentioned matrix comprising a solid support and a metal ion covalently attached thereto, or a matrix prepared by the above-mentioned method.

In an embodiment, the above-mentioned contacting is performed at a pH of about 4 to about 7, in a further embodiment at a pH of about 6.

In another embodiment, the above-mentioned contacting is performed in the presence of aspartic acid, glutamic acid, or a combination thereof.

In an embodiment, the above-mentioned elution is performed with a phosphate solution, in a further embodiment a sodium phosphate solution. In a further embodiment, the above-mentioned sodium phosphate solution is at a concentration of about 5 to about 200 mM.

In another embodiment, the above-mentioned proteolytic enzyme is an enzyme having endopeptidase activity, in a further embodiment an enzyme having trypsin-like activity.

In an embodiment, the above-mentioned matrix is in a chromatography column.

In an embodiment, the above-mentioned buffer is a 2-(N-morpholino)ethanesulfonic acid (MES) buffer. In another embodiment, the above-mentioned denaturing agent is urea.

In an embodiment, the above-mentioned phosphate solution further comprises acetonitrile.

In an embodiment, the above-mentioned method for obtaining a sample enriched in phosphorylated peptides from a protein-comprising sample further comprises labelling the phosphorylated peptides of (c) with a tag, such as an isotopic tag. In a further embodiment, the above-mentioned isotopic tag is an N-terminal tag. In another embodiment, the above-mentioned isotopic tag comprises deuterium (D) or carbon-13 (¹³C). In a further embodiment, the above-mentioned isotopic tag is pentafluorophenyl-4-anilino-4-oxobutanoate-D0 or pentafluorophenyl-4-anilino-4-oxobutanoate-D5.

In an embodiment, the above-mentioned two-dimensional liquid chromatography is performed using a weak anion exchange (WAE) column and a reverse phase (RP) column.

In an embodiment, the above-mentioned kit further comprises instructions setting forth the above-mentioned method for preparing an affinity matrix comprising a solid support and a metal ion covalently attached thereto.

In another aspect, the present invention provides a chromatography column comprising the above-mentioned affinity matrix.

In another aspect, the present invention provides a kit comprising the above-mentioned affinity matrix and a container.

In an embodiment, the above-mentioned kit further comprises instructions for purifying phosphorylated biomolecules from a sample.

In an embodiment, the above-mentioned phosphorylated biomolecule is a phosphoprotein or phosphopeptide.

Other objects, advantages and features of the present invention will become more apparent upon reading of the following non-restrictive description of specific embodiments thereof, given by way of example only with reference to the accompanying drawings.

### BRIEF DESCRIPTION OF DRAWINGS

In the appended drawings:

FIG. 1 shows an overview of an embodiment of the method of the present invention, for the detection of phosphorylation sites using two-step enrichment of phosphoproteins and phosphopeptides on iminodiacetic acid Sepharose™ resin with immobilized Al³⁺ ions (IDAS-Al resin). Protein samples were solubilized in a protein-denaturing buffer and loaded on columns with IDAS-Al resin. After washing with a high salt buffer phosphoproteins are eluted with 50 mM sodium phosphate, concentrated, denatured, treated with proteolytic enzymes, and the peptide digest is loaded again onto a column with IDAS-Al resin. The retained phosphopeptides are eluted as above with 50 mM sodium phosphate and resolved by 2-dimensional nanoflow liquid chromatography (2D-LC) on weak anion exchange (WAX) and reverse phase (RP) columns and then are identified and quantified by LC-MS/MS which also detects the phosphorylation sites from the fragmentation spectra. The non-phosphorylated peptides found in the flow-through are also resolved by 2D-LC and identified by MS/MS to provide supportive information;

FIG. 2 shows the enrichment of phosphopeptides from a tryptic digest of the protein mixture containing α-casein, BSA, ovalbumin and trypsinogen. (A) HPLC chromatogram. (B) Examples of peptides identified in the IDAS-Al resin eluate. (C) MS/MS-fragmentation spectrum of the α-casein phosphopeptide DIGsEsTEDQAMEDIK (SEQ ID NO: 3). The phosphorylated residues (phosphosites) are indicated in lowercase in the peptide sequences;

FIG. 3 shows the enrichment of phosphoproteins on IDAS-Al resin. (A) Recovery of radioactively labelled phosphoproteins from a total cell lysates. 1.4 mg of the total lysate of HeLa cells metabolically labelled with [³²P]-phosphate was loaded on a column containing 1 ml of IDAS-Al. The column was washed with three 1.5 ml portions of 30 mM MES, 1 M NaCl, pH 6, and phosphoproteins were eluted with three 0.5 ml portions of 20 mM phosphate buffer, pH 7. The graph shows average enrichment of specific radioactivity (cpm/mg of protein) in the combined eluates 1-3 was >6-fold. (B) Comparative enrichment of phosphoproteins from HeLa cell lysates using IDAS-Al column (SAL) and Qiagen™ PhosphoProtein purification kit under native and denaturing conditions. 5 μg of total protein from a total cell lysates (L), 2.25 μg of total protein from column flow-through (FT) and 0.25 μg of total protein from column eluate (E) fractions were resolved by SDS-PAGE. The gels were stained with phosphospecific Pro-Q™ Diamond phosphoprotein stain (upper panel) or silver stain (lower panel). Gels show a representative image of 3 independent experiments;

FIG. 4 shows the quantification of phosphopeptides by isotopic tagging using N-terminal SIMPL labels. (A) Extracted Ion Chromatograms of the bovine a-casein phosphopeptide VPQLEIVPNsAEER (SEQ ID NO: 1) (500 fmol) before and after modification with heavy and light SIMPL labels. The phosphorylated residue (phosphosite) is indicated in lowercase in the peptide sequence. SIMPL labelling only slightly reduces the intensity of the peptide spectra. (B) Validation of protein quantification. Tryptic peptides of bovine α-casein modified with the light and heavy SIMPL tags were mixed in the 1:9-9:1 ratio and analyzed by LC-MS/MS. The graph shows correlation between the expected protein ratios and those calculated from the ratio of the areas under EIC for peptides labelled with the heavy and light tags;

FIG. 5 shows the synthesis and structure of affinity resins comprising aluminum for purification of phosphoproteins and/or proshopeptides. (A) Synthesis of AI(OH)₂-Sephadex™ G25. Sephadex™ is treated with triethylalkyl aluminum, Al₂(Et)₆, dissolved in hexane (1.7 ml per g of Sephadex™ G25) followed by the addition of 0.06 ml of water. Then the gel is washed with diethyl ether and dried under vacuum. (B) Preparation of Iminodiacetyl-Al-Sepharose™. Iminodiacetyl-Sepharose™ is treated with a 50 mM solution of AlCl₃, which provides a resin containing Al³⁺ ions chelated with iminodiacetyl group;

FIG. 6 shows aluminum leakage from the resin. Aluminum leakage under different pH was measured for the AI(OH)₂-Sephadex™ resin and a commercially available IMAC(Al³⁺) resin (Amersham Chelating Sepharose Fast Flow™ charged with Al³⁺ ions). Concentration of Al³⁺ ions was measured by 8-oxychinoline assay and represented as nmol*ml⁻¹*mg⁻¹.

FIG. 7 shows the yield of phosphopeptides purification on Al(OH)₂-Sephadex™ G25 as a function of the pH of the loading buffer. Phosphopeptide concentrations in eluate are shown as percentage of the corresponding amounts in the sample before enrichment. The phosphorylated residues (phosphosites) are in brackets in the peptide sequences. Left dotted bars=TVDMES(pT)EVFTK (SEQ ID NO: 6), middle empty bars=DIG(pS)E(pS)TEDQAMEDIK (SEQ ID NO: 3), right hatched bars=VPQLEIVPN(pS)AEER (SEQ ID NO: 1);

FIG. 8 shows mass spectra of the phosphopeptide eluates after phosphopeptide enrichment of BSA and casein digests on Al(OH)₂-Sephadex™ G25 without (A) and with (B) addition of glutamic acid and aspartic acid in the loading buffer. Signals corresponding to phosphopeptides are marked with arrows. (C) Proposed mechanism of effect of glutamic acid and aspartic acid based on results of studies described herein: without being bound to any particular theory, it is believed that Glu/Asp may bind to the medium (Al(OH)₂-Sephadex™) and decrease interactions between the media and non-phosphorylated peptides, which in turn increases the selectivity of phosphoenrichment;

FIG. 9 shows an estimation of phosphopeptide capacity of Al(OH)₂-Sephadex™ resin. 30 μg aliquots of casein digest were sequentially loaded on 50 mg of Al(OH)₂-Sephadex™ resin and the amount of phosphopeptides in the flow-through fractions was measured by mass spectrometry. Results show that 50 mg of the resin binds a high fraction of the phosphopeptides from 150 μg of casein digest. The phosphorylated residues (phosphosites) are in brackets in the peptide sequences. Dashed line+triangles=TVDMES(pT)EVFTK (SEQ ID NO: 6), full line+black squares=DIG(pS)E(pS)TEDQAMEDIK (SEQ ID NO: 3), dashed line+empty squares=VPQLEIVPN(pS)AEER (SEQ ID NO: 1), dotted line+“x” signs=NMAINP(pS)KENLCSTFCK (SEQ ID NO: 7), dotted line+empty circles=YKVPQLEIVPN(pS)AEER (SEQ ID NO: 8);

FIG. 10 shows the peptides detected in the eluate after phosphopeptide enrichment of complex peptide sample (proteins from whole liver lysate digested with trypsin) using the Al(OH)₂-Sephadex™ resin. Accession numbers (Acc#) correspond to that from the UniProt database. The phosphorylated residues (phosphosites) are indicated in lowercase in the peptide sequences;

FIG. 11 shows Western blots depicting the enrichment of protein markers for LDS, (PLIN and HSL), and depletion of markers for cytoplasm (GAPDH), ER (calreticulin), mitochondria (cytochrome C), cytoskeleton (actin), lysosomes (LAMP-1) and PM (annexin I) in the RapiGest™ extracts of lipid droplets (LDE1) as compared to the total WAT homogenates (HG). 20 μg of total protein from HG, and 2 μg of total protein from LDE1 (A) or 10 μg of total protein from each fraction (B) was loaded on each lane. Re-extraction of the sample with equal volume of Laemmli sample buffer (LDE2) did not produce measurable amount of protein. The Western blotting was performed as described in Example 1;

FIG. 12 shows the application of isotope tagging and enzymatic dephosphorylation to measure the ratio between phosphorylated and non-phosphorylated forms of proteins. Proteins are digested with trypsin and then divided into 2 equal aliquots that are modified with either light or heavy isotope tags. One aliquot is dephosphorylated by alkaline phosphatase. Finally, the peptide mixtures are combined and LC-MS/MS analysis is performed. Non-phosphorylated peptides are detected as isotope pairs with equal intensities, whereas peptides that were phosphorylated are detected as single peaks and partially phosphorylated peptides are detected as doublets of unequal size;

FIG. 13 shows the correlation between theoretical and experimentally measured stoichiometry of phosphorylation for the bovine casein peptide (K)YKVPQLEIVPNsAEER(L) (SEQ ID NO: 8). Native and dephosphorylated casein as well as their mixtures in molar ratios from 1:9 to 9:1, were denatured and digested with trypsin. Peptides were separated to 2 equal aliquots and modified with the light and heavy SIMPL tags. One part was further dephosphorylated by alkaline phosphatase. Then the peptides were combined and the mixture was analyzed by LC-MS/MS. The stoichiometry of phosphorylation was calculated from the ratio of the areas under EIC for peptides labeled with the heavy and light tags. The graph shows correlation between the measured and expected protein ratios. The phosphorylated residue (phosphosite) is indicated in lowercase in the peptide sequence, and the flanking residues are between parentheses;

FIG. 14 shows the reproducibility of protein identification between three biological replicas. 383 not-redundant proteins were identified by at least 2 unique peptides with FDR<0.05. 227 proteins were identified in all three replicas, 95 were found in 2 replicas and 61 proteins were identified only in one replica of three;

FIG. 15 shows the distribution of isotope ratios for the subset of non-phosphorylated peptides lacking serine, threonine and tyrosine residues (A) and dependence of peptide ratios on the intensity of the peptide signals in MS spectra (B, left axis). Graph on panel B shows dependence of average deviation of isotope ratios (right axis) on the intensity of MS spectra;

FIG. 16 shows the quantification of PLIN phosphorylation by isotope tagging and enzymatic dephosphorylation. Panels show typical examples of MS-clusters, extracted ion chromatograms (EIC) and isotope ratios for the PLIN peptide ⁴⁰⁹LSLMEPESEFRDIDNPSAEAER⁴³⁰ (SEQ ID NO: 41) from WAT tissue of CL-treated (A) and control (B) mice; and

FIG. 17 shows the distribution of the biological functions of identified LDS proteins. The biological function (relation to a general molecular function and/or to a general biological process) was assigned to each protein using automated “slim” GO (gene ontology terms) annotation at NCBI NIH or manually assigned basing on the PubMed indexed literature data.

### DISCLOSURE OF INVENTION

Described herein is a matrix suitable for affinity-based purification and enrichment, the preparation thereof, and uses thereof.

In a first aspect, the present invention provides a matrix comprising a solid support and a metal ion covalently attached thereto. The matrix may be used for separation/purification/enrichment of molecules, such as biomolecules, such as phoshporylated biomolecues, such as phosphorylated polypeptides.

In another aspect, the present invention provides a matrix comprising a solid support and (i) aluminum, (ii) gallium, or (iii) any combination of (i) and (ii), covalently attached thereto. The matrix may be used for separation of phoshporylated biomolecues, such as phosphoproteins/phosphopeptides, e.g., as a phospho-affinity matrix.

In another aspect, the present invention provides a method for preparing a matrix comprising a solid support and a metal ion covalently attached thereto, said method comprising:

(a) contacting a solid support comprising hydroxyl groups attached thereto with an organometallic compound that is soluble in an organic solvent; and

(b) contacting the solid support of (a) with a source of free hydroxyl group.

In another aspect, the present invention provides a method for preparing a matrix comprising a solid support and (i) aluminum, (ii) gallium, or (iii) any combination of (i) and (ii) covalently attached thereto, said method comprising:

(a) contacting a solid support comprising hydroxyl groups attached thereto with an organometallic compound comprising (i) aluminum, (ii) gallium, or (iii) any combination of (i) and (ii), that is soluble in an organic solvent; and

(b) contacting the solid support of (a) with a source of free hydroxyl group.

The term “solid support” generally refers to chromatrographic media (e.g., resin, gel, beads) that are generally used to separate molecules and macromolecules based on various properties (e.g., size, charge, affinity for a given ligand, etc.). Solid supports comprising hydroxyl groups attached thereto are well known in the art and includes, for example, matrices of polysaccharides such as cross-linked polysaccharides. Examples of solid supports comprising hydroxyl groups attached thereto include Poly([allyl dextran]-co-N,N′-methylenebisacrylamide) (Sephacryl™), cross-linked dextran (e.g., Sephadex™), cross-linked agarose (e.g., Sepharose™). In an embodiment, the solid support is Sephadex™. In a further embodiment, the solid support is Sephadex™ G25. In an embodiment, the above-mentioned contacting is performed under conditions which are substantially free of water and/or oxygen.

The term “metal ion” refers to ions that are derived from, for example, simple salts (e.g., AlCl₃, NiCl₂, etc.), complex or mixed salts comprising both organic and inorganic ligands and metal complexes. Metal ions of use in practicing the present invention include, for example, main group metal ions, transition metal ions, lanthanide ions, etc. Zero valent metal precursors are included in this definition. In an embodiment, the metal ion has affinity for specific moieties, such as polyhistidine moieties (e.g., nickel, copper) or phosphorylated moieties (e.g., gallium, aluminum, iron, lead, mercury, nickel, cadmium, thallium, antimony, silver, chromium, manganese, platinum, gold, bismuth, iron, copper, zinc, cobalt, molybdenum, selenium, vanadium, calcium, Eu, Gd, Th, Sm, and so forth).

In an embodiment, the metal ion has a valency of 2 or 3 (i.e., 2⁺ or 3⁺ oxidation state). In an embodiment, the metal ion is Ga³⁺, Al³⁺, Fe³⁺, Co³⁺, Eu³⁺, Gd³⁺, Sm³⁺ and/or Tb³⁺. In a further embodiment, the metal ion is Al³⁺.

Organometallic compounds generally refers to chemical compounds containing bonds between carbon and a metal. Examples of organometallic compounds include alkylates of metal such as trialkyl metals (e.g., trialkyl aluminum, trialkyl gallium, etc.). In an embodiment, the above-mentioned trialkyl is a C₁-C₅ trialkyl (e.g., trimethylalkyl, triethylalkyl). In a further embodiment, the organometallic compound is triethylalkyl aluminum or gallium.

In an embodiment, the above-mentioned organic solvent is a non-polar solvent, such as hexane. In another embodiment, the above-mentioned source of free hydroxyl groups is water.

In an embodiment, the above-mentioned method further comprises (c) washing the matrix with a second organic solvent (e.g., diethyl ether and/or hexane).

In an embodiment, the above-mentioned method further comprises (d) drying the matrix. The matrix may be dried (i.e., evaporation of the solvent) using any method known in the art. In an embodiment, the above-mentioned drying is performed under vacuum.

In an embodiment, the leakage of Al³⁺ ions from said matrix (the concentration of Al³⁺ ions in the flow through) is less than about 0.2 nmol*ml⁻*mg⁻¹, in a further embodiment less than about 0.1 nmol*ml⁻¹*mg⁻¹, at pH values between about 5 to about 8, as measured by a 8-oxychinoline assay.

The matrix may be used in a number of different applications, with representative applications include phosphorylated biomolecule purification/enrichment applications, such as phosphoprotein/phosphopeptide purification applications. The term “phosphorylated biomolecules” includes, e.g., DNA, RNA, proteins, peptides, lipids and polysaccharides. Exemplary macromolecules include phosphorylated peptides, phosphorylated proteins, nucleic acids, oligonucletoides, polynucelotides, and phospholipids. Purification methods provided by the present invention generally include contacting a sample containing or suspected of containing a phosphorylated biomolecule, such as phosphoproteins/phosphopeptides, with an amount of the above-mentioned matrix under conditions which favor binding of the biomolecule to the matrix (i.e., to the covalently attached metal ions of the matrix), and then recovering (e.g., via elution with a suitable solution, such as a phosphate solution) the resultant bound phosphorylated biomolecule. One or more washing steps may optionally be included to remove undesirable components of the sample contacted to the matrix.

Accordingly, in another aspect, the present invention provides a method for obtaining a sample enriched in phosphorylated peptides from a protein-comprising sample, said method comprising:

(a) contacting the protein-comprising sample with a matrix comprising a metal ion having a 3⁺ oxidation state (e.g., aluminum, gallium, or a combination thereof).

(b) eluting the proteins from said matrix to obtain a phosphoprotein enriched sample;

(c) incubating the phosphoprotein enriched sample of (b) with a proteolytic enzyme thereby to obtain peptides;

(d) contacting the peptides of (c) with a matrix comprising a metal ion having a 3³⁰ oxidation state (e.g., aluminum, gallium, or a combination thereof); and

(e) eluting the peptides from the matrix of (d);

thereby obtaining a sample enriched in phosphorylated peptides.

When combined with well-known methods of mass spectrometry, the above-mentioned method facilitates separation of phosphopeptides from mixtures and facilitates their detection by mass spectrometry as well as sequencing of the peptides by tandem mass spectrometry. Methods well-known in the art may be applied to identify phosphoproteins in a sample from the sequences of phosphopeptides detected in a sample. When combined with methods for differential isotopic labelling, the methods of the invention can be employed to quantify relative amounts of phosphopeptides and phosphoproteins in different samples. These quantitative methods allow a comparison of phosphorylation state in samples derived from different sources (e.g., from different cell types), samples that are differentially affected by a treatment or stimulus (e.g., contact with a compound, a drug), by a change in environment (e.g., nutrient level) or by a change in condition or cell state (e.g., disease state, malignancy, site-directed mutation, gene knockouts) of a cell, tissue or organism from which the sample originated. The phosphoproteins identified in such a screen can function as markers for the changed state. Phosphopeptides and phosphoproteins from any naturally-occurring environment or artificially-controlled environment can be assessed by the methods herein. The method can be applied to mixtures of naturally-occurring proteins or peptides, as well as to mixtures of proteins or peptides derived from recombinant or synthetic methods.

Samples that can be analyzed by the above-mentioned methods include cell homogenates; cell fractions; biological fluids including urine, blood, and cerebrospinal fluid; tissue homogenates; tears; feces; saliva; lavage fluids such as lung or peritoneal lavages; mixtures of biological molecules including proteins, lipids, carbohydrates and nucleic acids generated by partial or complete fractionation of cell or tissue homogenates.

The methods provide for purification of a phosphoprotein(s)/phosphopeptide(s) from a sample which additionally contains other components, e.g., proteins other than phosphorylated proteins, and other non-protein components such as non-protein macromolecules. The starting sample is any sample containing or suspected of containing a phosphoprotein(s)/phosphopeptide(s) and one or more other components. In some embodiments, the phosphoprotein(s)/phosphopeptide(s) of interest is secreted into the culture medium in which cells are grown. In other embodiments, the phosphoprotein(s)/phosphopeptide(s) remains intracellular (e.g., in the cytoplasm, in a cell membrane, or in an organelle), in which case the cells are disrupted. A variety of protocols for disrupting cells to release an intracellular protein are known in the art, and can be used to extract a phosphoprotein from a cell. Such protocols are found in numerous publications, including, e.g., Current Protocols in Molecular Biology, (F. M. Ausubel, et al., Eds. 1987, and updates). Whether cell culture medium (“culture supernatant”) or disrupted cells (“cell lysate”) are used as the starting material, the starting material may be subjected to one or more treatments before being applied to a metal ion chelating resin. Such treatments include, but are not limited to, centrifugation, to remove cell debris, etc.; salt precipitation; application to a size exclusion chromatographic column; and application to an ion exchange chromatographic column.

In embodiments, the sample is obtained from an initial cellular composition by first combining the initial cellular composition with an amount of an extraction buffer. The extraction buffer may include an amount of a detergent sufficient to disrupt the formation of protein/protein complexes. The buffer is typically an aqueous composition that includes a suitable amount of a buffering agent, as is known in the art, where representative buffering agents include, but are not limited to: buffers, e.g., Tris-based buffers, borate-based buffers, phosphate-based buffers, imidazole, HEPES, PIPES, MOPS, PIPES, TES, MES and the like.

In an embodiment, the above-mentioned protein-comprising sample is a cell or tissue extract, and the method further comprises before (a): incubating said cell or tissue extract with a buffer comprising a denaturing agent. In another embodiment, the above-mentioned buffer comprising a denaturating agent is a 2-(N-morpholino)ethanesulfonic acid (MES) buffer. In a further embodiment, the above-mentioned denaturing agent is urea.

In an embodiment, the above-mentioned resin is an iminodiacetic acid-based resin having aluminum ions bound thereto. In a further embodiment, the above-mentioned iminodiacetic acid-based resin is Sepharose™.

In another embodiment, the above-mentioned matrix is a matrix prepared by the above-mentioned method.

In an embodiment, the above-mentioned elution is performed with a phosphate solution (e.g., a phosphate buffer, such as potassium or sodium phosphate). In a further embodiment, the above-mentioned phosphate solution is a sodium phosphate solution. In a further embodiment, the above-mentioned sodium phosphate solution is at a concentration of about 5 mM to about 200 mM. In another embodiment, the above-mentioned phosphate solution further comprises acetonitrile.

In an embodiment, the above-mentioned contacting is performed at a pH of about 4 to about 8. In a further embodiment, the above-mentioned contacting is performed at a pH of about 5 to about 7, in a further embodiment at a pH of about 6.

In another embodiment, the above-mentioned contacting is performed in the presence of glutamic and/or aspartic acid, such as in the presence of a 50 mM glutamic and/or aspartic acid solution.

In another embodiment, the above-mentioned method further comprises washing the column with a suitable buffer before one or more of the eluting steps. Such washing steps may be useful to remove any non-bound proteins or other sample constituents. One or more washing steps may be included, as desired to remove undesired components. In the broadest sense, the composition and temperature of a washing fluid may vary according to the desired result. Wash fluids may include a buffer, and may further include additional components, as necessary, including, but not limited to, a detergent. In an embodiment, the above-mentioned washing is performed with a MES buffer.

Peptide mixtures subjected to the above-mentioned method may be generated from natural or synthetic samples and may be the results of chemical, physical or enzymatic digestion of protein samples. Proteins can be digested using any enzymatic appropriate method, such as trypsin digestion. Peptides in the digest preferably range in size from about 10 to about 50 amino acids in length and are more preferably sized to facilitate peptide sequencing using tandem mass spectrometric methods. Those of ordinary skill in the art can select a protein digestion protocol suitable for use in protein sample(s) of interest.

In an embodiment, the above-mentioned proteolytic enzyme is trypsin or an enzyme having trypsin-like activity.

The above-mentioned matrix/resin may be provided in the form of a chromatography column, e.g., wherein the matrix is packed in a column. Accordingly, in another aspect, the present invention provides a chromatography column comprising the above-mentioned affinity matrix. The matrix may also comprise a structure that is a solid support of any shape or configuration. Thus, the resin can be in any form or utilized in any format, e.g., a bead, a sheet, a well, and the like. Where the resin is a bead, the beads may be provided in various sizes, depending, in part, on the nature of the sample being applied, where suitable bead sizes include from about 10 μm to about 500 μm, e.g., from about 10 μm to about 200 μm, from about 20 μm to about 150 μm, from about 50 μm to about 150 μm. Non-limiting examples of formats in which a matrix is provided include a gravity-flow column; a fast protein liquid chromatographic (FPLC) column; a multi-well (e.g., 96-well) column format; a spin column; and the like.

In embodiments, binding to the matrix may for example be achieved by column chromatography, batch treatment, or expanded bed absorption approaches. Column chromatography typically entails packing the solid matrix onto a chromatography column, passing the sample through the column to allow binding, passing a wash buffer through the column and subsequently an elution buffer to collect the bound material. Batch treatment typically entails combining the sample with the solid matrix in a vessel, mixing, separating the solid matrix (e.g., by centrifugation), removing the liquid phase, washing, separating the solid matrix (e.g., re-centrifuging), adding an elution buffer, separating the solid matrix (e.g., re-centrifuging) and removing the eluate. In expanded bed adsorption, the solid matrix particles are placed in a column where liquid phase is pumped in from the bottom and exits at the top. Hybrid approaches may also be used, for example binding via a batch method followed by packing the solid matrix with the bound target molecule onto a column, followed by washing and elution on the column.

In an embodiment, the above-mentioned method further comprises labelling the phosphorylated peptides of (c) with an isotopic tag. Proteins and/or peptides in different samples can be differentially isotopically labeled to facilitate comparison of amounts of the same peptide or protein in different samples. The methods of the present invention may employ protective groups which are isotopically labeled to generate pairs or sets of reagents that are substantially chemically identical, but which are distinguishable by mass. For example a pair of protective group reagents, one of which is isotopically heavy and the other of which is isotopically light can be employed for the comparison of two samples, one of which may be a reference sample containing one or more known proteins in known amounts. For example, any one or more of the hydrogen, nitrogen, oxygen or sulfur atoms in the protective group may be replaced with their isotopically stable isotopes: ²H (deuterium), ¹³C, ¹⁵N, ¹⁷O, ¹⁸O or ³⁴S. Methods for the isotopic labelling of peptides are well known in the art (see, for example, WO/2005/012247 and Fedjaev et al. Rapid Commun Mass Spectrom. 2007; 21(16):2671-90). In an embodiment, the above-mentioned isotopic tag is an N-terminal tag. In another embodiment, the above-mentioned isotopic tag comprises deuterium (²H or D) or carbon-13 (¹³C). In yet another embodiment, the above-mentioned isotopic tag is pentafluorophenyl-4-anilino-4-oxobutanoate-D0 or pentafluorophenyl-4-anilino-4-oxobutanoate-D5.

In another aspect, the present invention provides a method for analyzing the phosphopeptide content of a sample comprising (i) separating the phosphopeptides comprised in the sample enriched in phosphorylated peptides obtained by the above-mentioned method by two-dimensional liquid chromatography, and (ii) identifying the separated peptides by mass spectrometry.

The sequence of a phosphopeptide and the identification of the site(s) of phosphorylation can be determined for example by a combination of tandem mass spectrometry and computer-assisted database search programs, such as SEQUEST™ (University of Washington, Seattle Wash.) (McCormack, A. L. et al. (1996), Anal. Chem. 69, 767-776; Eng, J. K. et al. (1994), J. Amer. Soc. Mass. Spectrom., 5: 976-989; U.S. Pat. No. 5,538,897 (Jul. 23, 1996) Yates, III et al.) or Spectrum™ Mill software (Rev. A.03.02.060a, Agilent Technologies). In the first stage of a tandem mass spectrometer, any given phosphopeptide is selected and subjected to collision induced dissociation (CID). The spectra of a resulting fragment ion is recorded in the second stage of the mass spectrometry, as a so-called CID spectrum. This process is repeated with other (ideally all) peptides present in the sample. Because the CID process usually causes fragmentation at peptide bonds and different amino acids for the most part yield peaks of different masses, a CD spectrum alone often provides enough information to determine a peptide sequence. Peptide sequencing and protein identification is facilitated by using a sequence searching computer program which takes all known genomic sequences, computes all possible theoretical CID spectra and compares them to experimental CID spectra for matches and sequence identification. The mass modification to the N-terminus, C-terminus, glutamic acid, aspartic acids and any other acidic side groups are known and this information can be incorporated into the computer analysis. Also mass changes due to phosphorylation are also known and can be incorporated into the computer analysis. Data can be searched for any possible phosphorylations to serine, tyrosine, and threonine residues, thus allowing the identification of sites of phosphorylation.

In an embodiment, the above-mentioned two-dimensional liquid chromatography is performed using a weak anion exchange (WAE) column and a reverse phase (RP) column.

In another aspect, the present invention provides a method for determining the ratio of phosphorylated to non-phosphorylated forms of a polypeptide comprising:

(a) contacting a sample comprising said polypeptide with a proteolytic enzyme to obtain peptides;

(b) labelling a first fraction of the peptides of (a) with a first isotopic tag and a second fraction of the peptides of (a) with a second isotopic tag, wherein said first and second isotopic tags have different masses;

(c) contacting said first or second fraction with a dephosphorylating agent;

(d) submitting an equivalent amount of said first and second fraction to mass spectroscopy to obtain a mass spectroscopy profile; and

(e) determining the ratio of phosphorylated to non-phosphorylated forms of said polypeptide in accordance with the mass spectroscopy profile obtained.

In another aspect, the present invention provides kits and systems comprising the above-mentioned matrix, which may be used, for example, to practice the above-mentioned methods. The kits and systems at least include the matrix described above. The kit may alternatively comprises a solid support (e.g., a solid support comprising hydroxyl groups such as Sephadex™) together with reagents to covalently attach a metal ion to the solid support. In embodiments the kit may comprise the matrix in a dry form, in a form suspended in a solvent, in a suitable vessel/container or in a device directly conducive to use in a purification/separation method (e.g., pre-packed in a column or centrifuge tube). The kits and systems may also include a number of optional components that find use in the subject methods. Optional components of interest include buffers, including extraction/loading/washing buffer or buffers, and the like. In embodiments, the kits will further include instructions for practicing the subject methods. These instructions are typically printed on for example a package insert, the packaging, reagent containers, combinations thereof, and the like.

### MODE(S) FOR CARRYING OUT THE INVENTION

The present invention is illustrated in further details by the following non-limiting examples.

### EXAMPLE 1

### Materials and Methods

Reagents. All reagents were purchased from Sigma-Aldrich except where indicated otherwise. All reagents were of the highest grades commercially available and all solutions were prepared using HPLC-grade solvents. Synthesis of isotopic labels has been performed as previously described (Fedjaev M, et al., Rapid Commun Mass Spect, 2007, 21(16): 2671-2679), except aniline-1,2,3,4,5,6-¹³C (4.9 g, Cambridge Isotope Laboratories) was used instead of aniline-2,3,4,5,6-d5.

Preparation of iminodiacetic acid-Al³⁺-Sepharose (IDAS-Al) resin. 0.5 ml of Chelating Sepharose Fast Flow™ (GE Healthcare) gel slurry was placed into Poly-Prep™ chromatography column (Bio-Rad) and washed with two 0.5 ml portions of water, 1 ml of 50 mM aluminum chloride (AlCl₃) and 1.5 ml of water.

Preparation of Al(OH)₂ Sephadex™ resin. 5.3 grams of Sephadex™ G25 fine media (Amersham Biosciences) was mixed with 20 ml of absolute hexane and 9 ml of a 1 M solution of triethylaluminum (9 mmol) in hexane was added drop-wise within 30 min with constant stirring. After 30 min, the reaction was terminated by addition of 0.32 ml of water. The product was filtered, washed with diethyl ether and hexane and dried under vacuum. Product yield was 5.73 grams (98%).

Measurement of aluminum concentration with 8-oxychinoline assay. Samples (250 μl) eluted from the resins were diluted with 500 μl of 200 mM acetate buffer (pH=5.5) and mixed with 750 μl of 1% (w/v) 8-oxychinoline in chloroform. The mixture was vortexed for 10 min, and then organic phase was separated and its absorbance at 395 nm was measured. The concentration of aluminum ions was calculated using a calibration curve established with standards of AlCl₃ of known concentrations.

Tryptic digestion of model proteins. Bovine alpha-casein, chicken egg albumin (ovalbumin) and BSA were dissolved at the concentration of 2 mg/ml in 50 mM ammonium bicarbonate buffer (pH 7.8) and treated with trypsin (Promega sequencing grade) for 18 h at 37° C. (protein/trypsin ratio=50/1). The resulting peptide mixture was desalted using C18 spin columns (Harvard Apparatus) according to the manufacturer's protocol, dried using a SpeedVac™ apparatus (Savant) and stored at −20° C. until further use.

Enrichment of phosphopeptides from the mixture of purified protein standards. Purified protein standards: alpha-casein (20 μg), bovine serum albumin (BSA) (60 μg) and ovalbumin (20 μg) were dissolved in 100 μl of 200 mM MES, pH 6, and loaded on a microspin column (Harvard Apparatus) containing 150 μl of IDAS-Al resin equilibrated with two 200 μl portions of 100 mM MES, pH 6 containing 1 M NaCl. The column was centrifuged at 2,000×g for 2 min. The flow-through fraction was collected and reapplied onto the same column. The resin was then washed with three 200 μl fractions of 100 mM MES, pH 6, and peptides were eluted with three 100 μl fractions of 50 mM phosphate, pH 7.5, containing 10% acetonitrile (ACN). Proteins from eluates, washes and flow-through fractions were digested with trypsin and analyzed by LC-MS/MS as described below.

Labelling and quantification of purified bovine α-casein. Bovine α-casein was dissolved in 50 mM ammonium hydrocarbonate to the final concentration of 5 μg/μl mixed with sequencing grade modified trypsin (Promega; trypsin to protein ratio 1 to 100) and incubated overnight at 37° C. Tryptic peptides were desalted on Microspin™ columns filled with C18-silica (Harvard Apparatus) according to manufacturer protocol, dried on Speedvac™ and labelled with isotopic tags as previously described (Fedjaev M, et al., supra). The peptides labelled with light and heavy tags were dissolved in 200 mM MES buffer pH 6, mixed in different ratios (10:0, 9:1, 8:2, 7:3, 6:4, 5:5, 4:6, 3:7, 2:8, 1:9, 0:10) and loaded onto MicroSpin™ columns containing 150 μl portions of IDAS-Al gel for the enrichment of phosphopeptides as described above.

Proteins were resuspended in 50% trifluoroethanol (TFE) containing 50 mM ammonium bicarbonate and 10 mM dithiotrietol (DTT) at a final concentration of 1 mg/ml and incubated for 5 minutes at 90° C., then treated with iodoacetamide (final concentration 25 mM) for 40 minutes in the dark. Excess of iodoacetamide was quenched by the addition of DTT to a final concentration of 2.5 mM. After evaporation of TFE with a SpeedVac™, proteins were precipitated with 5% TCA and resuspended in 20 mM ammonium hydrocarbonate, pH 7.8, containing sequencing grade trypsin (Promega, trypsin/protein ratio 1:100). Digestion was performed overnight at 37° C. Twenty μl of each sample (˜20 μg of protein digest) was mixed with 35 μl of a 0.5 M solution of diisopropylethylamine in acetonitrile, 25 μl of a 1 M solution of 4-anilino-4-oxobutanoic acid (d0 or d5) in dimethylformamide and 20 μl of a 0.3 M solution of pentafluorophenyl-4-anilino-4-oxobutanoate (d0 or d5) in acetonitrile. The mixtures were incubated for 3 hours at 37° C. and the excess of unreacted label was quenched by the addition of 50 μl of water. The pairs of samples labelled with the heavy (d5) and light (d0) tags were combined, acidified with 5% formic acid to the final concentration of 0.2% and 4-anilino-4-oxobutanoic acid was removed by triple extraction with ethyl acetate (300 μl). Alternatively, desalting can be performed by gel filtration on 0.5 ml Sephadex™ G-10 column connected to an Agilent™ 1100 Series liquid chromatography system.

Metabolic labeling and purification of phosphoproteins from cultured HeLa cells. HeLa cells (American Type Culture Collection, Rockville, Md.) were grown at 37° C. under 5% CO₂ in MEM (GIBCO-BRL, Grand Island, N.Y.) containing 1% penicillin and 1% streptomycin, 1% MEM nonessential amino acids (GIBCO-BRL) and 10% decomplemented FBS (GIBCO-BRL). Cultures were split (1:6) when they reached 75-90% confluence. For metabolic labelling of phosphoproteins, cells were incubated for 15 min in phosphate-free Dubecco's Modified Eagles Medium (D-MEM, Invitrogen), and for 3 h, in the same medium supplemented with [³²P]-phosphate (ICN, Irvine, Calif.), 0.1 mCi/ml. The radioactive medium was then removed, cells were placed on ice, washed twice with ice-cold PBS and lysed for 30 min in a 50 mM MES buffer pH 6, containing 6 M urea (lysis buffer). The sample was then centrifuged at 13,000×g for 10 min at 4° C. to remove cell debris and protein concentration in the lysate was measured using the Bradford assay kit from Bio-Rad. Aliquots containing 5 mg of total protein were diluted with the lysis buffer to the final volume of 10 ml and loaded on spin columns containing 0.5 ml of IDAS-Al gel. Columns were washed with five 1-ml aliquots of lysis buffer containing 1 M NaCl and phosphoproteins were eluted with four 0.5 ml aliquots of 50 mM sodium phosphate, pH 7.5.

Extraction and analysis of soluble phosphoproteins from mouse liver (Example 3). All animals were maintained in the Canadian Council on Animal Care (CCAC)-accredited animal facilities of the CHU Ste-Justine Research Center. Four month-old (˜25-29 g BW) male C57BL/6J mice, were purchased from Charles River Canada (St-Constant, PQ, Canada), housed in an animal facility with 12:12-h light-dark cycles at 25° C., and fed ad libitum with a standard mouse chow. Animals fed or fasted overnight (18 h) were sacrificed by cervical dislocation. Livers were immediately removed and homogenized in ice-cold 0.25 M sucrose buffer (5 mM Tris-HCl buffer, pH 7.4, containing 0.25 M sucrose, 1 mM benzamidine, 1 mM PMSF, 1 mM MgCl₂, 2 mM NaF, and 2 mM sodium orthovanadate; 4 ml of buffer per 1 g of liver) using a Potter-Elvehjem™ homogenizer with 12 strokes of a motorized Teflon™ pestle at 1,500 rpm. The homogenates were centrifuged at 35,300 g for 1 h at 4° C. and aliquots of supernatants containing 5 mg of total protein were diluted to final volume of 10 ml with 6 M urea, 50 mM MES pH 6.0. A 0.5 ml aliquot of diluted fraction was used for enrichment of phosphoproteins on IDAS-Al resin as described above. Phosphoproteins were precipitated by trichloroacetic acid (TCA, final concentration 10% v/v).

Protein pellets were washed with ice-cold acetone and resuspended in 50% trifluoroethanol (TFE) containing 50 mM ammonium bicarbonate and 10 mM dithiotrietol (DTT), incubated for 5 minutes at 90° C., and alkylated with iodoacetamide (final concentration 25 mM) for 40 minutes in the dark. Excess of iodoacetamide was quenched by the addition of DTT to a final concentration of 2.5 mM. After evaporation of TFE using a SpeedVac™, proteins were digested with trypsin as described above.

Peptides were desalted on spin columns filled with silica C18 media (Harvard apparatus) and labelled with pentafluorophenyl-4-anilino-4-oxobutanoate-d0 or pentafluorophenyl-4-anilino-4-oxobutanoate-d5 (SIMPL isotopic tags) as previously described (Fedjaev M, et al., supra). Labelled peptides from starved and fed mice were combined and used for phosphopeptide enrichment on Microspin™ columns each containing 50 μl of IDAS-Al resin as described above. Both eluates containing phosphopeptides and flow-through fractions containing non-phosphorylated peptides were analyzed by 2D-LC-MS/MS.

Extraction and analysis of soluble phosphoproteins from mouse liver (Example 7). C57-black mice, 8 weeks of age (20-25 g body weight), were purchased from Charles River Canada (St. Constant, PQ, Canada), housed in an animal facility with 12:12-h light-dark cycles at 25° C., and fed ad libitum with Purina™ chow. Animals were killed by decapitation. The livers were homogenized in ice-cold 50 mM MES, 6 M urea buffer pH=6 (4 ml of buffer per 1 g of liver) using a Potter-Elvehjem™ homogenizer with 6 passes of a motorized Teflon pestle at 1,500 rpm. The homogenate was centrifuged at 13,000 g for 10 min at 4° C. Proteins were reduced with 5 mM dithiothreitol for 30 min at 37° C. and thiogroups were alkylated with 12 mM iodoacetamide for 30 min in the dark at room temperature. Then sample was diluted six times with 50 mM ammonium hydrocarbonate and proteins were digested with trypsin (sequencing grade, Promega, protein:enzyme mass ratio=50:1) overnight at 37° C.

Offline WAX HPLC of peptides. Pre-MS fractionation of peptides was done by HPLC combining concentrating and desalting of peptides on RP-C18 column and their separation on a weak anion exchange (WAX) column. Each sample was loaded on C18 (Jupiter™ 4 um Proteo 90 A 50×1 mm, Phenomenex) column connected to an Agilent™ 1100 Series LC system. The column was washed with 20 mM ammonium acetate containing 10% ACN and peptides were eluted with 20 mM ammonium acetate containing 70% ACN directly onto the WAX column (PolySulfoethyl A 1000×1 mm 5 μm 300 A). The WAX column was washed with 20 mM ammonium acetate, 10% ACN for 10 min and peptides were eluted with 0-100% linear gradient of 2 M ammonium formate, 10% ACN at a speed of 20 μl/min. Thirty 50 μl fractions were collected acidified with formic acid and analyzed by pLC-MS/MS.

pLC-MS/MS analysis. Peptide digests were analyzed in duplicates using a LC-MS/MS instrument consisting of an Agilent™ 1100 Series nanoflow liquid chromatography system and LC/MSD-Trap-SL™ ion trap mass spectrometer (Agilent Technologies, Santa Clara, Calif.). The peptides were enriched on a Zorbax™ 300SB-C18 trap column (5 μm, 5×0.3 mm) and separated by reversed phase chromatography on a Zorbax™ 300SB-C18 analytical column (3.5 μm, 150×0.075 mm, Agilent) column (10×0.075 mm) packed with Biobasic™ C18 with a gradient of 5-90% acetonitrile in 0.1% formic acid at a flow rate of 200 or 300 nl/min. The column eluate was sprayed directly into the mass spectrometer. Acquisition parameters were as following: (a) positive mode, (b) scan range from 400 to 2200 m/z, (c) scan speed of 13000 m/z×sec⁻¹, (d) “trap drive” of 90, (e) MS/MS of a maximum 3 most intense ions per cycle, (f) preferred charge state+2, (g) active exclusion after 2 spectra for 1 min. Spectra were recorded in a profile mode.

The mass spectra were interpreted and quantified using the Spectrum™ Mill software (Rev. A.03.02.060a, Agilent Technologies). Using a “Data Extractor” module of the program, the MS/MS spectra were (a) converted to a centroid mode based on the area in the top 50% of a peak, (b) merged by similarity (in order for spectra to be merged, at least 25 out of their 50 most intensive peaks had to match and the matching peaks had to represent >70% of the total spectral intensity) within a time window of ±40 sec and precursor m/z window ±1.2 and (c) filtered by quality (precursor signal to noise ratio >25, sequence tag length >1). For each precursor the MS intensity was calculated as the area under the extracted chromatograms of ion intensity versus the retention time (EIC) using the same parameters as for the MS/MS spectra merging (±40 sec, ±1.2 m/z). The ratios between the light and heavy forms of labelled peptides were calculated by comparing the MS intensities for the ions having the mass difference of 5 Da and presumably representing the isotopomers, which identity was further confirmed by MS/MS of at least one of these ions.

“MS/MS search” was performed for the 25 most intensive peaks in every spectrum using the rodent or human subsets of UniProt™ database (release of Sep. 7, 2006; Nucleic Acids Res. 2008, 36 (Database issue): D190-5. Epub 2007 Nov. 27; ftp://ftp.expasy.org) with the following parameters:

For Examples 2-6: (a) tryptic digestion, 1 maximum missed cleavage, (b) appropriate mass shifts for the modified amino acids (e.g. N-terminus: 4-anilino-4-oxobutanoate (d0/d5) or unmodified) (c) precursor mass tolerance ±2.5, fragment mass tolerance ±0.7, (d) minimum 6 detected peaks. Then for the peptides for which the predicted MS/MS peaks could contribute for more then 70% of the total intensity of the experimental spectrum the Spectrum Mill scores were calculated based on the following bonus-penalty system. Bonuses were given for the assigned peaks according to their fragment type (e.g., y- and b-type ions had score of 1, a-type ions, 0.25, etc.) and penalties were given for unassigned peaks proportionally to their intensities. For each peptide the score was also calculated using the “decoy” database containing reversed protein sequences (Moore R E, et al., Am. Soc. Mass Spectrom., 2002, 13: 378; Peng J, et al. J. Proteome Res., 2003, 2: 43). For each charge the rate of false positive identifications (FP, the ratio between the number of total identifications in the decoy database and in the target database) was calculated as a function of the score. The peptide score cut-off was set to yield the FP <0.05 and only the peptides for which the difference in the scores obtained by the searches in the target and “decoy” databases was more then 1.5 (2 for the peptides with the charge of +4) were selected for further analysis. In order to address the minimal requirements of the protein identification guidelines (Taylor, G K and Goodlett, D R. Rapid Commun. Mass Spectrom. 2005 19: 3420) for the proteins that were identified by one peptide only an additional protein score (the sum of the maximum scores of the non-redundant peptides) cut-off was set to 12 to further reduce the FP identifications in this category.

For Example 7: (a) tryptic digestion, a maximum of one missed cleavage, (b) precursor mass tolerance ±2.5, fragment mass tolerance ±0.7, (c) variable modifications included phosphorylation of serine, threonine and tyrosine residues and fixed modifications included carbamidomethylation of cysteine residues, (d) tryptic digestion with at most 1 missed enzymatic cleavage per peptide. Then the Spectrum Mill scores were calculated based on the following bonus-penalty system. Bonuses were given for the assigned peaks according to their fragment type (e.g., y- and b-type ions had score of 1, a-type ions, 0.25) and penalties were imposed on unassigned peaks in proportion to their intensities.

Purification of lipid droplets (LD) from mouse white adipose tissue (WAT). All animals were maintained in the Canadian Council on Animal Care (CCAC)-accredited animal facilities of the CHU Ste-Justine Research Center. Two month-old (˜25-29 g BW) male C57BU6J mice, were purchased from Charles River Canada (St. Constant, PQ, Canada), and housed for an additional month at 25° C. in an animal facility with 12:12 h light-dark cycles. During this time, animals were fed ad libitum with 5K chow (˜18% protein, ˜6% fat, ˜5% fiber). Animals were injected peritoneally with 1 mg/kg BW of β-adrenergic receptor agonist CL316,243 (CL, (disodium 5-[(2R)-2-[[(2R)-2-(3-Chlorophenyl)-2-hydroxyethyl]amino]propyl]-1,3-benzodioxole-2,2-dicarboxylate, Sigma) or sham-injected with saline and sacrificed by cervical dislocation 15 min later. WAT was immediately removed and homogenized in the ice-cold homogenization buffer (150 mM Tris, pH 7.4, 50 mM Na₃VO₄, 50 mM NaF, 10 mM EDTA, 1 mM PMSF, complete protease inhibitor cocktail (Sigma) and a Roche PhosSTOP™ phosphatase inhibitor cocktail), using 10 ml of buffer per 1 g of WAT, in a Potter-Elvehjem™ homogenizer with 12 passes of a motorized Teflon pestle at 1,500 rpm. The homogenate was filtered through a nylon mesh and centrifuged at 17 g for 2 min. Pellet and infranatant were removed and the floating lipid droplets resuspended in 2 ml of the homogenization buffer and centrifuged as above. After the second wash with 50 mM TRIS, pH 7.4, containing 1 M NaCl (Fisher), the LD were snap-frozen and kept at −80° C. until further use.

Extraction of LD proteins and preparation of peptide digests. Purified LD preparations (average wet weight 250 mg) were resuspended in 1 ml of ice-cold acetone, sonicated for 25 sec at 80 W, incubated for 16 h at −20° C. and centrifuged for 10 min at 13,000 g. The supernatants were discharged and pellets were re-extracted twice with ice-cold acetone (1 h each time). After removal of residual acetone under a stream of nitrogen, the protein pellets were re-suspended in 300 μl of a 0.1% sodium 3-[(2-methyl-2-undecyl-1,3-dioxolan-4-yl)methoxyl]-1-propanesulfonate (RapiGest™ SF) in 50 mM NH₄HCO₃, pH 7.8, containing 5 mM DTT, then sonicated for 10-25 sec and incubated in a boiling bath for 5 min. After 5-min centrifugation at 13,000 g, supernatants were treated for 30 min with IAA in the dark (final concentration 15 mM), and digested overnight with trypsin (Promega, sequencing grade; trypsin to protein ratio 1:50). After digestion, samples were supplemented with TFA (final concentration 0.5%), incubated at 37° C. for 1 h and centrifuged at 13,000 g for 10-min to remove RapiGest™ SF.

Labeling and dephosphorylation of LD peptides. Peptide digests (˜200 μg of protein) were de-salted by the reverse phase chromatography on macro-C18 spin columns (Harvard apparatus) eluted with 70% acetonitrile in water, dried in a SpeedVac™ Concentrator (Savant) and labeled with heavy or light SIMPL (Stable Isotope-containing Multiplex Peptide Labels) isotope tags as described (Fedjaev et al., 2007, supra). After adjusting pH to 8.0 with 500 mM NH₄HCO₃, samples were supplemented with 3 units of bovine intestinal alkaline phosphatase (BIAP, Sigma, P-5521) and incubated for 3 h at 37° C. BIAP reaction was terminated by addition of concentrated TCA (Sigma) to a final concentration 10%, followed by 1 h incubation on ice and 30-min centrifugation at 13,000 g. Samples containing dephosphorylated peptides were then mixed 1:1 with untreated samples, extracted twice with ethylacetate (Sigma) to remove the excess of unreacted SIMPL labels and analyzed by 2D-LC-MS/MS as described below.

Enrichment of LD phosphopeptides on iminodiacetic acid Sepharose with immobilized Al³⁺ ions. For detection of phosphorylation sites, tryptic digests of LD proteins prepared as described above were loaded on a spin columns containing Sepharose™ modified with iminodiacetic acid (Chelating Sepharose Fast Flow™, GE Healthcare Life Sciences) charged with Al³⁺ ions equilibrated with 100 mM Bis-Tris buffer, pH 6.0 (50 μl of resin per 200 μg of peptide digest). The columns were washed with 3 volumes of 100 mM Bis-Tris buffer, pH 6.0 containing 1 M NaCl and eluted with 3 portions (one total column volume each time) of 100 mM sodium phosphate buffer, pH 7.4.

HPLC of peptides on weak anion exchange (WAX) column. Offline WAX HPLC was performed on an Agilent™ 1100 Series liquid chromatography system. Peptides were loaded on C18 (Jupiter™ 4 μm Proteo 90 Å 50×1 mm, Phenomenex) column in 10 mM ammonium acetate containing 10% ACN, and eluted with step gradient of 70% of ACN directly on the WAX (PolySulfoethyl A 1000×1 mm 5 μm 300 Å) column. The column was washed with 10 mM ammonium acetate buffer containing 10% ACN for 10 min and peptides were eluted with 0-100% gradient of 2 M ammonium formate, 10% ACN. Thirty five (25) fractions of 50 μl were collected. Collected fractions were immediately acidified with FA (final concentration 0.2%) and analyzed by LC-MS/MS.

μLC-MS/MS analysis of LD peptides. Peptides were analyzed using a LC-MS/MS instrument consisting of an Agilent™ 1100 Series nanoflow liquid chromatography system and LC/MSD-Trap-SL™ ion trap mass spectrometer (Agilent Technologies, Santa Clara, Calif., USA). The peptides were enriched on a Zorbax™ 300SB-C18 trap column (5 μm, 5×0.3 mm) and separated by reversed-phase chromatography on a Zorbax™ 300SB-C18 analytical column (3.5 μm, 150×0.075 mm, Agilent) with a gradient of 5-90% acetonitrile in 0.1% formic acid at a flow rate of 300 μl/min. The column eluate was electrosprayed directly into the mass spectrometer. Acquisition parameters were as following: (a) positive mode, (b) scan range from 400-2200 m/z, (c) scan speed of 13000 m/z xs-1, (d) ‘trap drive’ of 90, (e) MS/MS of a maximum three most intense ions per cycle, (f) preferred charge state +2, (g) active exclusion after two spectra for 1 min.

Phosphopeptides enriched on iminodiacetic acid Sepharose™ with immobilized Al³⁺ ions were analyzed on 4000 Q TRAP™ mass spectrometer (Applied Biosystems). The peptides were enriched on a Zorbax™ 300SB-C18 trap column (5 μm, 5×0.3 mm) and separated by reversed phase chromatography on a Zorbax™ 300SB-C18 analytical column (3.5 μm, 150×0.075 mm, Agilent) or PicoFrit™ (New Objective) column (10×0.075 mm) packed with Biobasic™ C18 with a gradient of 5-90% acetonitrile in 0.1% formic acid at a flow rate of 200 or 300 nl/min. The column eluate was sprayed directly into the mass spectrometer. Acquisition parameters for the LC/MSD-Trap-SL were as following: (a) positive mode, (b) scan range from 400 to 2200 m/z, (c) scan speed of 13000 m/zxsec-1, (d) “trap drive” of 90, (e) MS/MS of a maximum 3 most intense ions per cycle, (f) preferred charge state +2, (g) active exclusion after 2 spectra for 1 min. Spectra were recorded in a profile mode. For the LC/4000 Q TRAP′ instrument the spectra were acquired with: (a) positive mode, (b) scan range for MS from 350 to 1600 m/z, for MS/MS 70 to 1700 m/z , (c) scan speed of 4000 m/z×sec⁻¹, (d) enhanced resolution scan at 250 m/z×sec-1, (e) MS/MS of a maximum 3 most intense ions per cycle, (f) charge states: +2, +3, +4, (f) dynamic exclusion of 1.5 min.

Data processing. The mass spectra were interpreted and quantified using Spectrum Mill™ software (Rev. A.03.03.073, Agilent Technologies). Using a “Data Extractor” module of the program, the MS/MS spectra were (a) converted into a centroid mode based on the area in the top 50% of a peak, (b) merged by similarity (in order for spectra to be merged, at least 25 out of their 50 most intensive peaks had to match and the matching peaks had to represent >70% of the total spectral intensity) within a time window of ±40 s and precursor m/z window ±1.2, and (c) filtered by quality (precursor signal-to-noise ratio >25, sequence tag length >1). For each precursor, the MS intensity was calculated as the area under the extracted chromatograms of ion intensity versus the retention time (EIC) using the same parameters as for the MS/MS spectra merging (±40 s, ±1.2 m/z). The ratios between the light and heavy forms of labelled peptides were calculated by comparing the MS intensities for the ions having the mass difference of 6 Da and presumably representing isotopomers. The identity of these peptides was confirmed by MS/MS of at least one of the ions.

MS/MS search was performed for the 25 most intensive peaks in every spectrum using the rodent subset of UniProt™ database with the following parameters: (a) tryptic digestion, a maximum of one missed cleavage, (b) appropriate mass shifts for the modified amino acids (e.g., N-terminus: 4-anilino-4-oxobutanoate or unmodified) (c) precursor mass tolerance ±2.5, fragment mass tolerance ±0.7. Then for the peptides for which the predicted MS/MS peaks could contribute for more than 70% of the total intensity of the experimental spectrum, the Spectrum MiII™ scores were calculated based on the following bonus-penalty system. Bonuses were given for the assigned peaks according to their fragment type (e.g., y- and b-type ions had score of 1, a-type ions, 0.25, etc.) and penalties were imposed on unassigned peaks in proportion to their intensities. For each peptide the score was also calculated using the “decoy” database containing reversed protein sequences. For each charge, the false discovery rate (FDR, the ratio between the number of total identifications in the target database and in decoy database) was calculated as a function of the score. The peptide score cutoff was set to keep FDR <0.05 and only the peptides for which the difference in the scores obtained by the searches in the target and ‘decoy’ databases was more then 1.5 (2 for the peptides with the charge of +4) were selected for further analysis. In order to address the minimal requirements of the protein identification guidelines, for the proteins that were identified by one peptide only an additional protein score (the sum of the maximum scores of the non-redundant peptides) cut-off was set to 12 to further reduce the FP identifications in this category.

For each biological replica, average isotope ratios between the BIAP-treated and untreated samples and standard deviations were calculated. The distribution of ratios in the sample provides information about their variability. For each isotope ratio a p value representing the likelihood that a value equal to or higher than the given ratio can be obtained randomly was calculated from the distribution of isotope ratios for non-phosphorylated peptides lacking the serine, threonine and tyrosine residues using GraphPad Prism™ software. Peptides with statistically significant changes in isotope ratios in two of three replicas were further validated by manual analysis of the MS spectra.

### EXAMPLE 2

### Consecutive Enrichment of Phosphoproteins and Phosphopeptides on Iminodiacetic Acid-Aluminum-Sepharose (IDAS-Al)

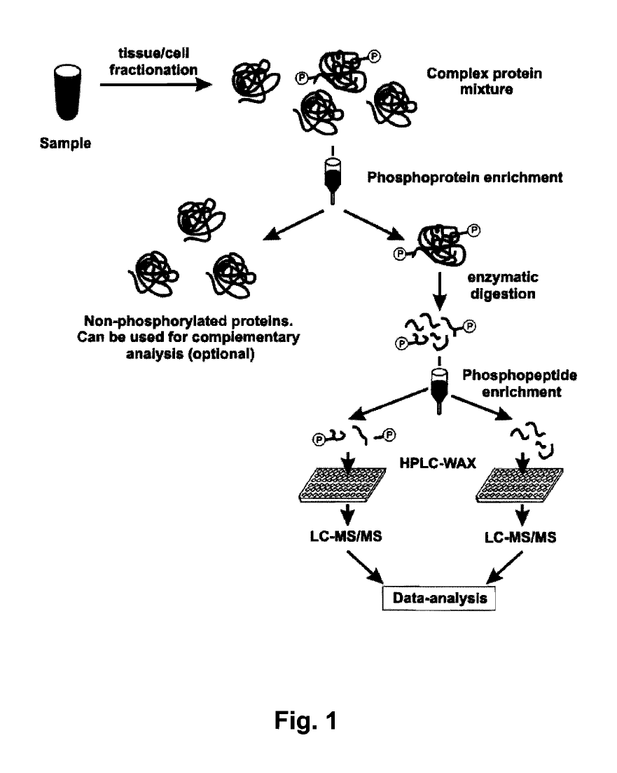

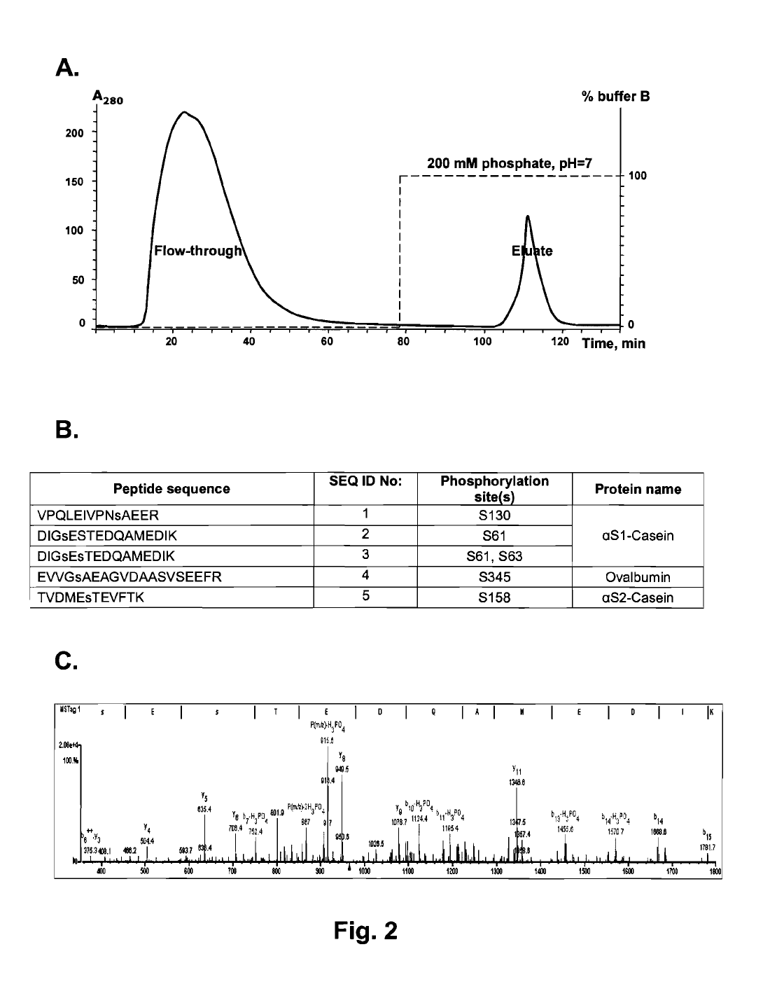

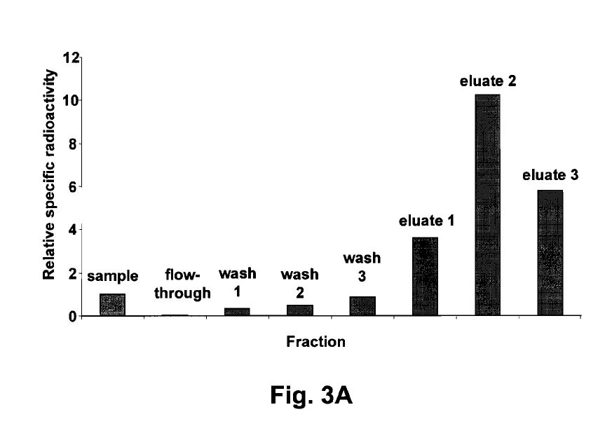

An effective method for the identification of phosphopeptides based on the two-step consecutive enrichment of phosphoproteins and phosphopeptides was developed (FIG. 1). First, cells, tissues or their fractions are solubilized in a buffer, containing a protein denaturing agent (such as 8M urea or 50% trifluoroethanol) and a detergent, are loaded onto a column containing IDAS-Al. After washing with a high-salt buffer phosphoproteins are eluted with 50 mM sodium phosphate, concentrated and treated with trypsin or similar proteolytic enzymes, and the peptide digest is loaded onto an IDAS-Al column. The retained phosphopeptides are eluted as above with 50 mM sodium phosphate, resolved by 2-dimensional liquid chromatography (2D-LC) on weak anion exchange (WAX) and reverse phase (RP) columns and then are identified by MS/MS which also detects the phosphorylation sites from the fragmentation spectra. The non-phosphorylated peptides found in the flow-through are also resolved by 2D-LC and identified by MS/MS to provide supportive information for protein identification. The described method has been first tested using mixtures of purified proteins containing bovine serum albumin and trypsinogen that do not contain phosphorylation sites and 2 phosphoproteins, ovalbumin, and bovine α-casein. It was found that a two-step enrichment of phosphopeptides on IDAS-Al column results in specific and quantitative purification of all phosphopeptides from the protein (FIG. 2). Using HeLa cells labelled with ³²P-phosphate, it was demonstrated that radioactively-labelled proteins were quantitatively bound to IDAS-Al column and eluted with phosphate. Phosphoproteins were enriched by about 6-fold as measured by the increase of specific radioactivity (FIG. 3A). To study further specificity of the procedure of phosphoprotein enrichment under denaturing and non-denaturing conditions, the Pro-Q™ Diamond Phosphoprotein Gel Stain (Invitrogen) specific for phosphopeptides was used (FIG. 3B). These experiments also showed that the method resulted in the extraction of all phosphoproteins from HeLa cell lysates, as detected using Pro-Q™ Diamond Phosphoprotein Gel Stain. Extraction was as complete as that achieved with a commercial phosphoprotein purification kit from Qiagen (Qiagen™ PhosphoProtein purification kit). However the commercial kit could not be used under denaturing conditions (FIG. 3B). The phosphoproteins from HeLa cells were further digested and used to compare efficacy of the phosphopeptide purification using IDAS-Al column with efficacy of the 2 leading methods for phosphopeptide purification: Ga³⁺ IMAC columns (Phosphopeptide Isolation Kit from Pierce Biotechnologies) and TiO₂ microspheres (Phos-trap™ Phosphopeptide Enrichment Kit from Perkin-Elmer). In duplicate experiments 75-90% of all detected peptides purified using IDAS-Al column were phosphopeptides as compared to the ˜40-50% enrichment achieved with the TiO₂ microspheres kit and the ˜50% enrichment obtained with Ga³⁺ IMAC Kit (Table I).

TABLE I
Comparison of Seph-Al (“SAL” in the table), Ga³⁺ IMAC (Phosphopeptide Isolation Kit
from Pierce Biotechnologies (“PI” in the table) and TiO₂ microspheres (Phos-trap ™
Phosphopeptide Enrichment Kit from Perkin-Elmer (“PE” in the table) for specificity of
phosphopeptide enrichment.

| | | | | | | Average | Average |
|---|---|---|---|---|---|---|---|
| | | | | | | enrichment | enrichment |
| | | | Non | | % of | for the | for the |
| | | Phospho | phospho | Total | phospho | same | same |
| Sample | Exper. | peptides | peptides | peptides | peptides | sample | method |
| SAL/SAL | 1 | 44 | 9 | 53 | 79 | 75 | 82 |
| | 2 | 40 | 16 | 56 | 71 | | |
| QI/SAL | 1 | 48 | 8 | 56 | 86 | 88 | |
| | 2 | 40 | 4 | 44 | 91 | | |
| SAL/PE | 1 | 2 | 2 | 4 | 50 | 45 | 47 |
| | 2 | 2 | 3 | 5 | 40 | | |
| QI/PE | 1 | 0 | 0 | 0 | 0 | 50 | |
| | 2 | 8 | 8 | 16 | 50 | | |
| SAL/SAL | 1 | 44 | 6 | 50 | 88 | 89 | 89 |
| | 2 | 39 | 4 | 43 | 91 | | |
| SAL/PI | 1 | 34 | 29 | 63 | 54 | 56 | 50 |
| | 2 | 40 | 28 | 68 | 59 | | |
| SAL/PI | 1 | 26 | 21 | 47 | 55 | 57 | |
| | 2 | 27 | 19 | 46 | 59 | | |
| QI/PI | 1 | 12 | 6 | 18 | 67 | 39 | |
| | 2 | 6 | 45 | 51 | 12 | | |

QI = Qiagen ™ PhosphoProtein purification kit.

### EXAMPLE 3

### Quantitative Analysis of the Proteome Using SIMPL Tags

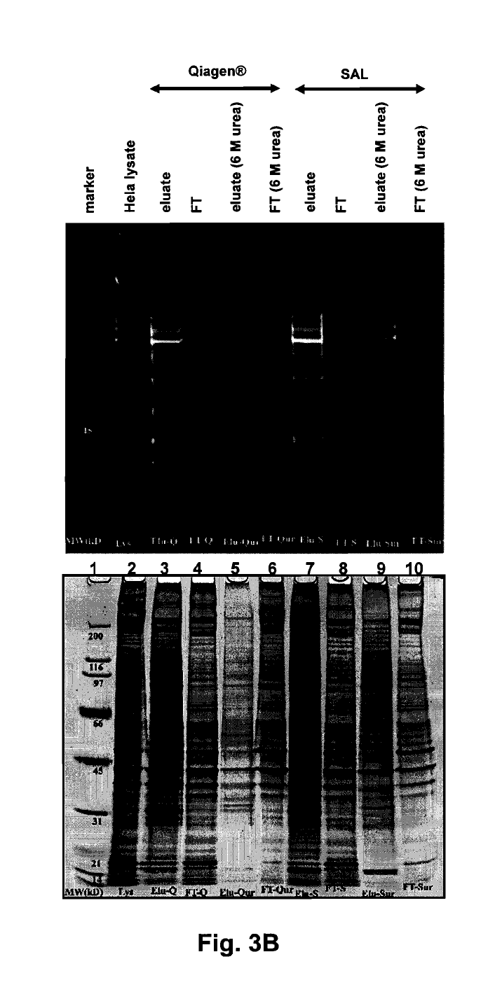

The quantitative analysis of the proteome was performed using a method based on the N-terminal labelling of tryptic peptides with so-called SIMPL tags consisting of pentafluorophenyl-4-anilino-4-oxobutanoate containing 0, 3 or 6 atoms of ¹³C or 0 or 5 atoms of deuterium (Fedjaev M, et al., supra). When labelling is performed in 250 mM diisopropylethylammonium 4-anilino-4-oxobutanoate buffer (pH 5.5) containing 55% (v/v) of acetonitrile and 25% (v/v) of dimethylformamide, SIMPL tags preferentially react with the terminal amino groups of peptides allowing effective comparison of protein/peptide abundance between samples. The labelling with SIMPL tags also improves the collision-induced fragmentation of the labelled peptides, increasing the efficiency of their identification from the MS/MS spectra and, as a result, the total protein coverage. The results presented herein show that SIMPL tags are fully applicable for the quantification of phosphopeptides. The intensity of the MS spectra for native phosphopeptides was compared to that of phosphopeptides modified with SIMPL labels (representative spectra are shown at FIG. 4A) and it was found that SIMPL labelling only slightly reduces the intensity of the peptide spectra.

Accuracy of quantifications was evaluated using the tryptic digests of purified bovine α-casein labelled with light and heavy SIMPL labels. The digests were mixed in the 1:9-9:1 ratio and loaded onto IDAS-Al columns for the purification of phosphopeptides. When the purified phosphopeptides were analyzed by LC-MS/MS, it was determined that the relative abundance of phosphopeptides was measured with an accuracy between 1 and 9% (FIG. 4B) which could potentially be improved using MS instruments having a higher resolution.

The above method was the applied to the analysis of complex protein mixtures, namely the comparison between cytosolic (i.e., soluble) phosphoproteins from livers of mice fed ad libitum and of mice that underwent an 18-hour starvation. Livers were rapidly homogenized and the phosphoproteins were purified, reduced/alkylated and digested with trypsin as described above (Example 1). After labelling with SIMPL tags, mixed phosphoprotein digests were loaded on IDAS-Al columns for purification of phosphopeptides, and both phosphopeptides and non-phosphopeptides were analysed by 2D-LC-MS/MS. Three biological replicas were analyzed for each state. The light and heavy labels were alternated between replicas in order to eliminate any possible effect due to the isotopes.

Using 5 mg of total soluble liver protein for each replica, an average of 20,750 MS/MS spectra were collected per sample, leading to the identification, with a 5% false-positive rate, of 2428 non-redundant phosphopeptides representing 948 phosphoproteins including low abundant receptors, transcription factors and kinases. On average, 74% of peptides purified by the double-enrichment procedure were phosphorylated according to the MS/MS data. The identification of more than 80% of the proteins was confirmed by the analysis of flow-through fraction containing non-phosphorylated peptides. 511 of the proteins identified were previously known to have phosphorylation sites, according to SwissProt™ annotations. These data were compared with those reported by Moser and White using high capacity IMAC and LC-MS/MS (Moser K, and White F M, J Proteome Res. 2006 5(1): 98-104) who identified 299 phosphopeptides representing 223 proteins in the rat liver. 92 peptides and 54 unique phosphosites identified by Moser and White were also identified in the studies described herein, demonstrating that our technology may lead to identification of phosphoproteins that have not been yet reported in the literature.

### EXAMPLE 4

### Synthesis and Optimization of the New Affinity Resin for Phosphopeptide Purification Based on Al(OH)₂-Sephadex™

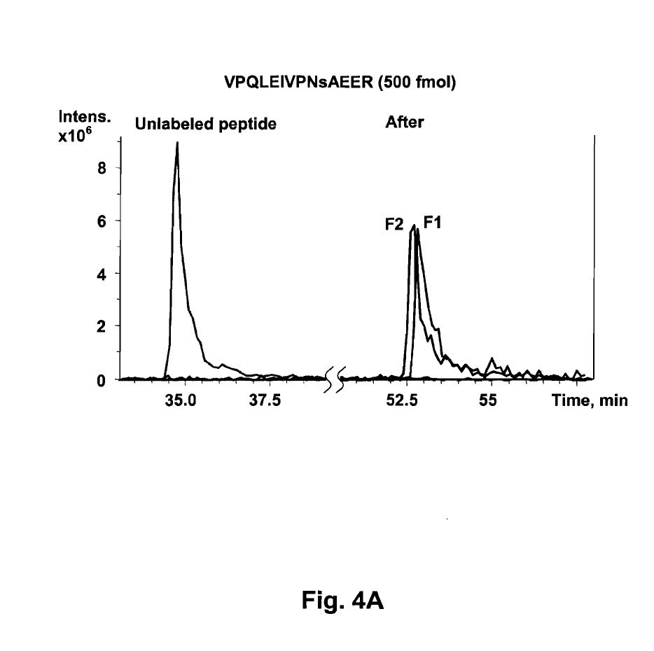

A new affinity chromatographic resin comprising Sephadex™ covalently modified with aluminum hydroxide was developed for the purification of phosphorylated peptides. The procedure for the synthesis of the Al(OH)₂-Sephadex™ is shown in FIG. 5. First hydroxyl groups on the surface of Sephadex™ are modified with triethyl aluminum in organic solvent in the absence of water and oxygen. This is followed by the addition of water which hydrolyses the remaining ethyl groups for hydroxyl groups.

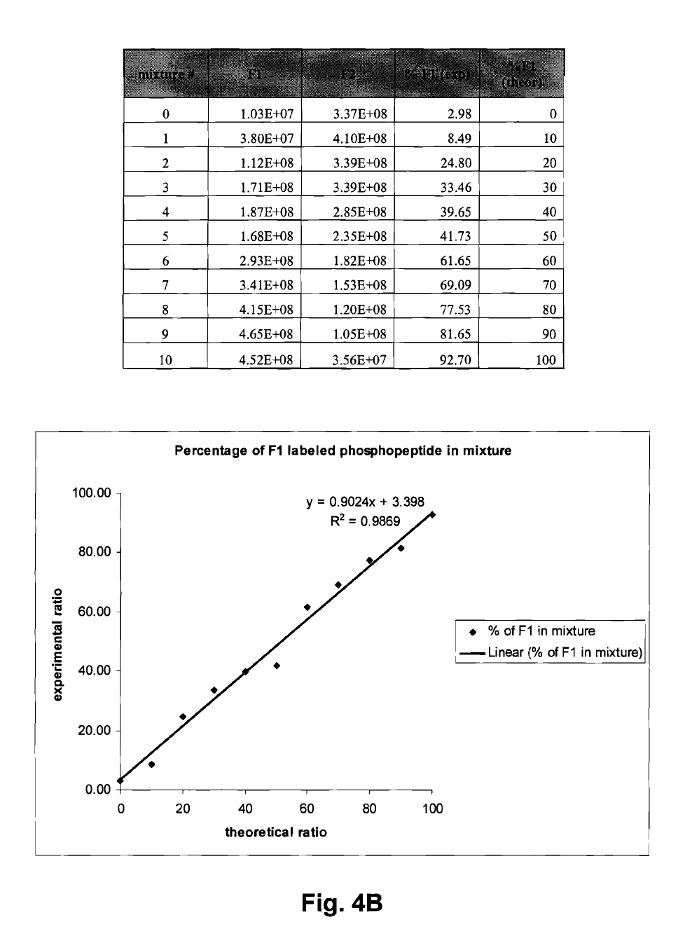

FIG. 6 shows that the aluminum leakage under different pH is lower for the Al(OH)₂-Sephadex™ resin as compared to a commercially available IMAC(Al³⁺) resin (Amersham Chelating Sepharose Fast Flow™ resin charged with Al³⁺ ions), despite the fact that Al(OH)₂-Sephadex™ resin contains about 18-fold more aluminum as compared to chelating Sepharose Fast Flow™ resin (1.33 mmol/g as compared to 0.074 mmol/g).

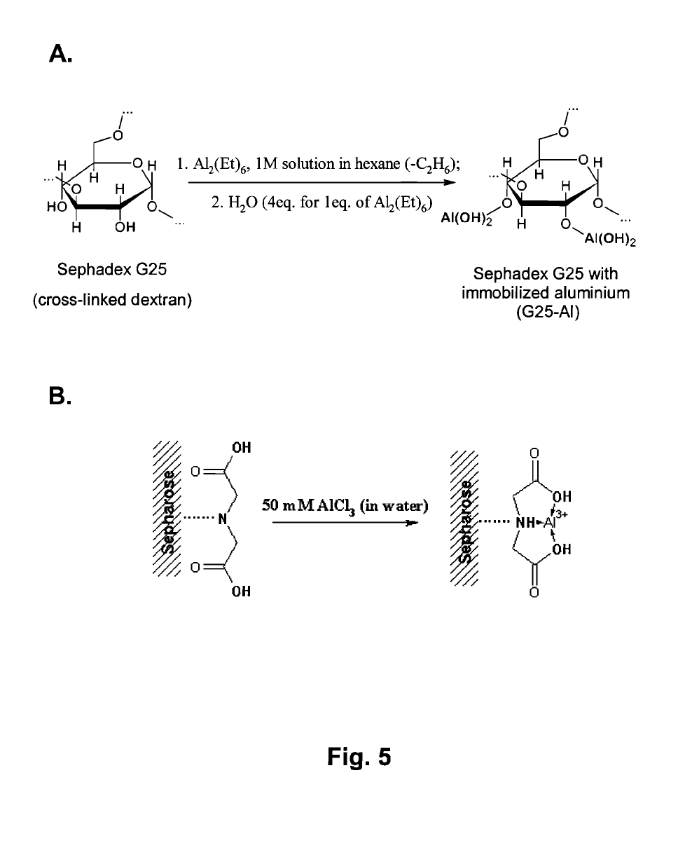

The binding of phosphorylated peptides to the Al(OH)₂-Sephadex™ resin was tested using the digest of purified alpha-casein at different pH between 2.0 and 8.0. In each case 20 ug of casein digest was loaded onto 10 mg of the resin and the mixture was incubated for 5 min. Then the resin was washed 3 times by the same buffer and eluted with 50 mM sodium phosphate pH 7.5. As shown in FIG. 7 significant amounts of phosphopetides can be retained on the resin at a pH varying from about 4 to about 7, with an optimal yield at a pH of about 6.

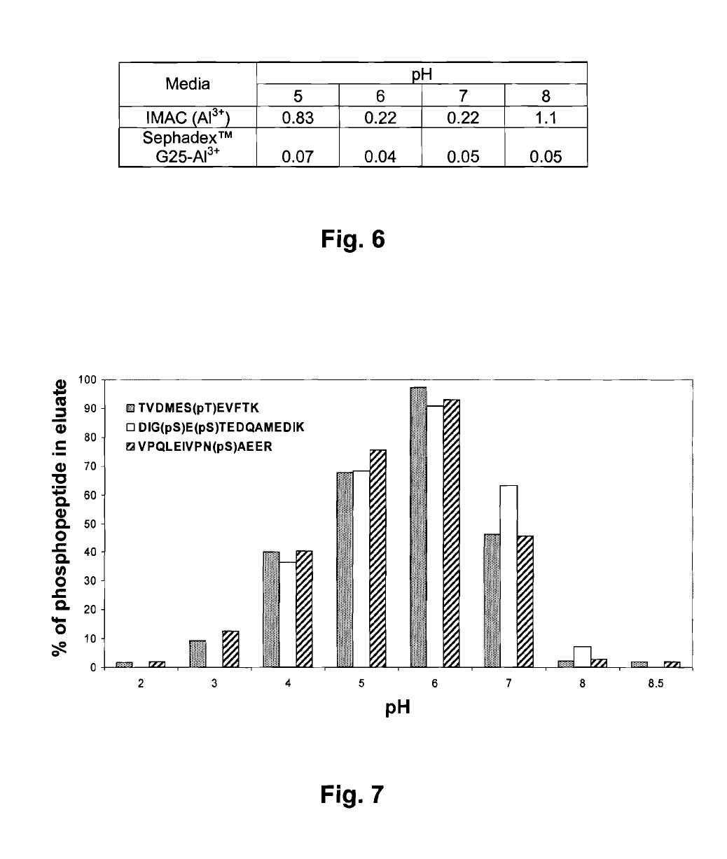

The specificity of binding was studied using the mixture of alpha-casein and BSA digests. FIG. 8 shows that the addition of aspartic acid and glutamic acid at a concentration of 50 mM in the loading buffer significantly reduces non-specific binding to the Al(OH)₂-Sephadex™ resin.

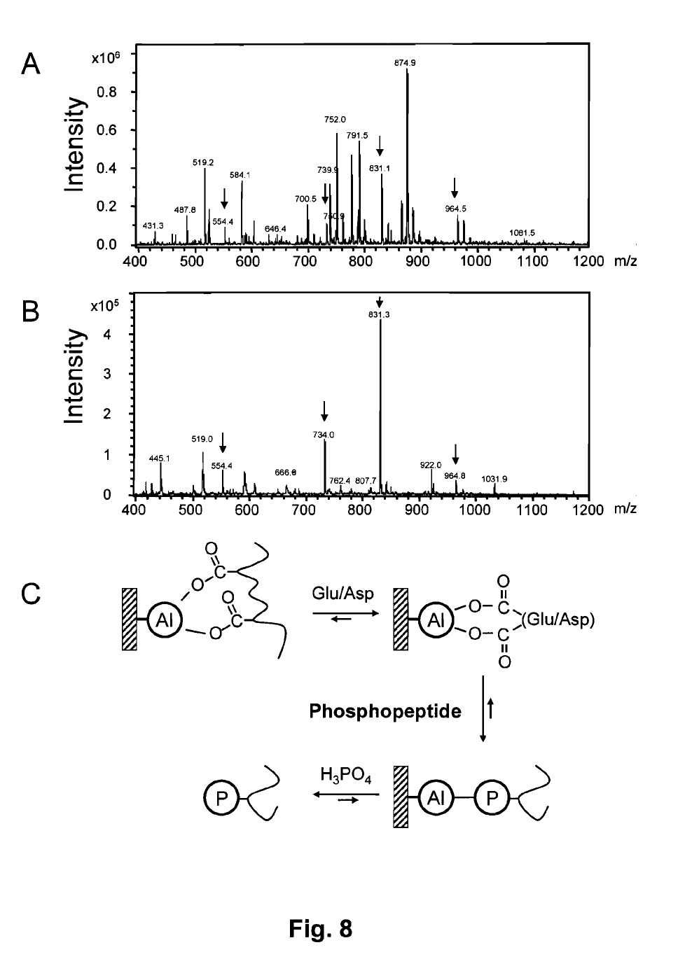

The capacity of the Al(OH)₂-Sephadex™ resin was tested using alpha-casein digest. Data presented at FIG. 9 shows that 50 mg of the resin binds a high fraction (˜>90%) of the phosphopeptides from up to 150 μg of casein digest (corresponding to about 0.7 nmol of phosphogroups), but appears to get gradually saturated when amounts greater than 150 μg are loaded on the resin.

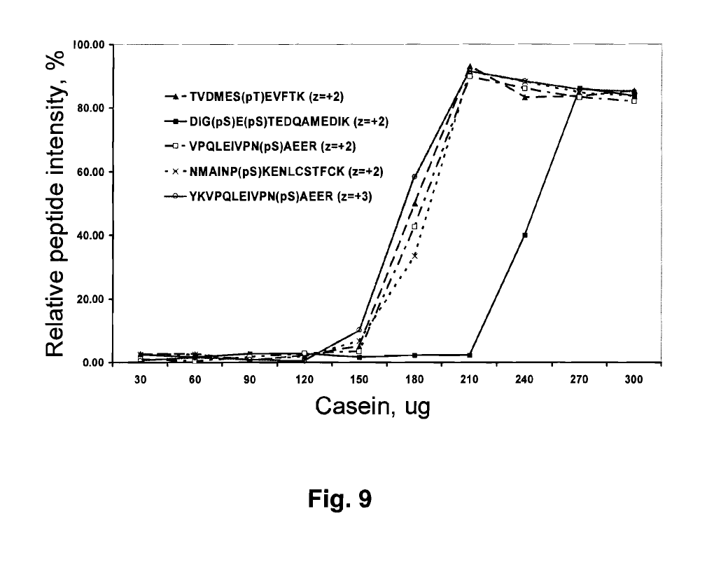

The efficacy of phosphopeptide purification on Al(OH)₂-Sephadex™ resin was further tested using the tryptic digest of cytosolic proteins from mouse liver. Livers were homogenized in a buffer containing 0.25 M sucrose and a complete cocktail of phosphatase and protease inhibitors. Soluble proteins were precipitated by TCA, denatured by RapiGest™ SF detergent and digested with trypsin. A sample containing a digest of 1 mg of total protein was loaded of Al(OH)₂-Sephadex™ resin. After elution with 50 mM phosphate, peptides were analysed by LC-MS/MS. FIG. 10 shows that the eluate after phosphopeptide enrichment of a whole liver lysate digested with trypsin using the Al(OH)₂-Sephadex™ resin comprises 26 different phosphopeptides containing 29 phosphorylation sites, as well as 6 non-specific peptides) which corresponds to a ˜80% efficacy of phosphopeptide enrichment.

### EXAMPLE 5

### Isolation of Lipid Droplets and Solubilization of Proteins

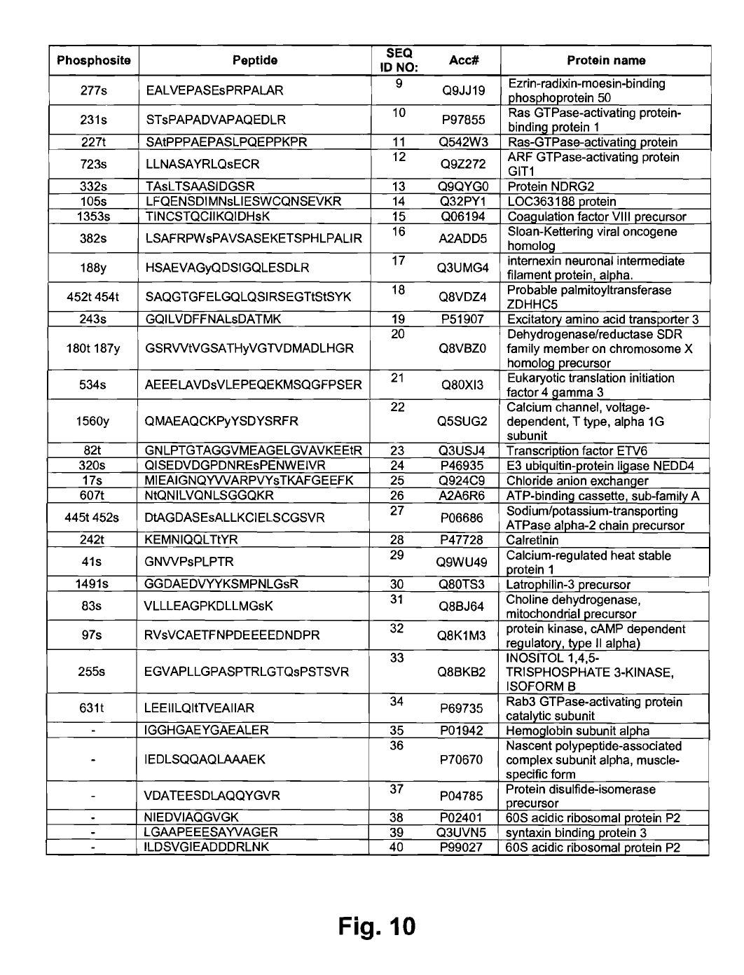

Three month-old (˜25-29 g BW) male C57BL/6J mice received peritoneal injection of either the β3-receptor agonist CL316,243 (CL) or saline. Fifteen minutes later, they were sacrificed by cervical dislocation, WAT tissue was removed and rapidly homogenized in a buffer containing inhibitors of proteases and phosphatases, followed by the purification of LD using low-speed centrifugation. LDs accumulated as a floating fat layer on the top of the infranatant containing soluble proteins and the pellet containing most cellular organelles. LDs were purified on average 5-fold compared to the tissue homogenate as estimated by Western blotting with PLIN and HSL as markers for LDs (FIG. 11A). Close degree of purification was detected for the tissues from both untreated and treated mice. The preparation was essentially free of the protein markers of plasma membrane (Annexin 1), lysosomes (LAMP-1), cytosol (GAPDH) and mitochondria (cytochrome C), but contained substantial amounts of the ER marker calreticulin (FIG. 11B and Table II).

TABLE II
Depletion of the markers for the cytoplasm, mitochondria, lysosomes,
plasma membranes and nuclei and enrichment of the markers for LDS
in the lipid droplet extracts as compared to total WAT homogenates.
Table shows representative data of 1 of 6 purifications all of which yielded
similar results.

| | | Enrichment |
|---|---|---|
| Marker | Organelle | (fold) |
| Perilipin | Lipid droplet | 8 ± 2 |
| HSL | Lipid droplet/cytosol | 6.5 ± 1.4 |
| GADPH | Cytosol | 0.2 ± 0.1 |
| Calreticulin | ER | 1 ± 0.3 |
| Cytochrome C | Mitochondria | <0.05 |
| Actin | Cytoskeleton | <0.1 |
| LAMP-1 | Lysosome | ND |
| Annexin-1 | Plasma membrane | ND |
| Na⁺/K⁺-ATPase | Plasma membrane | <0.1 |

For the solubilization of LD proteins, the following procedure was developed. After extraction of most lipids in cold acetone, the protein pellet was sonicated in a 0.1% (v/w) solution of RapiGest™ SF detergent, and then incubated for 5 min in a boiling bath. Analysis by SDS-PAGE followed by protein assay and Western blotting showed that RapiGest™ treatment resulted in solubilization of all measurable protein including the vast majority of PLIN and HSL (FIG. 11A).

### EXAMPLE 6

### Analysis of Stoichiometry of LD Protein Phosphorylation

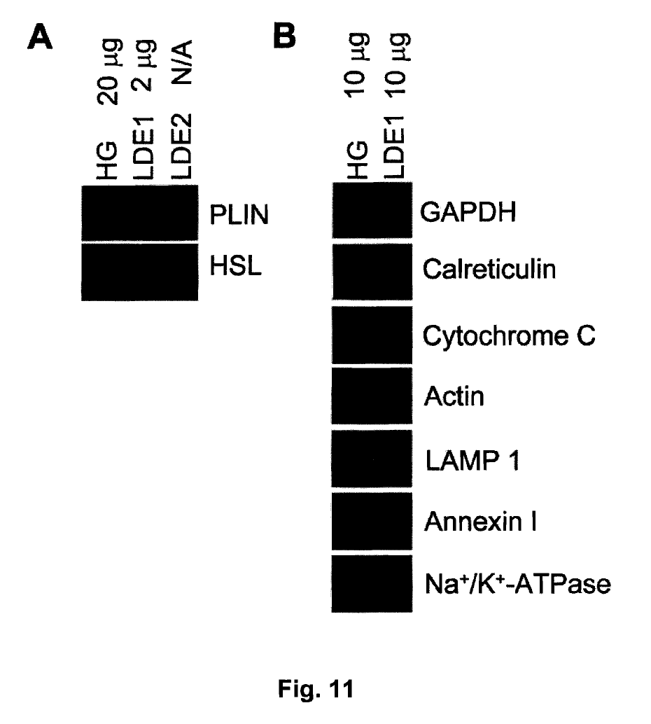

To analyse the stoichiometry of protein phosphorylation, a method that combines enzymatic dephosphorylation and global isotope tagging of peptides was applied (FIG. 12). In this method, proteins are denatured, digested and the resulting peptides are divided into equal aliquots which are further modified with light and heavy isotope tags, respectively. Peptides modified with one of the tags are further treated by bovine intestinal alkaline phosphatase (BIAP). Then both mixtures are combined and analyzed by LC-MS/MS. Peptides that do not contain phosphorylated residues are detected as isotope pairs of equal intensity, whereas peptides that initially were fully phosphorylated) are detected as single peaks. Partially phosphorylated peptides are detected as doublets of unequal size (FIG. 12).

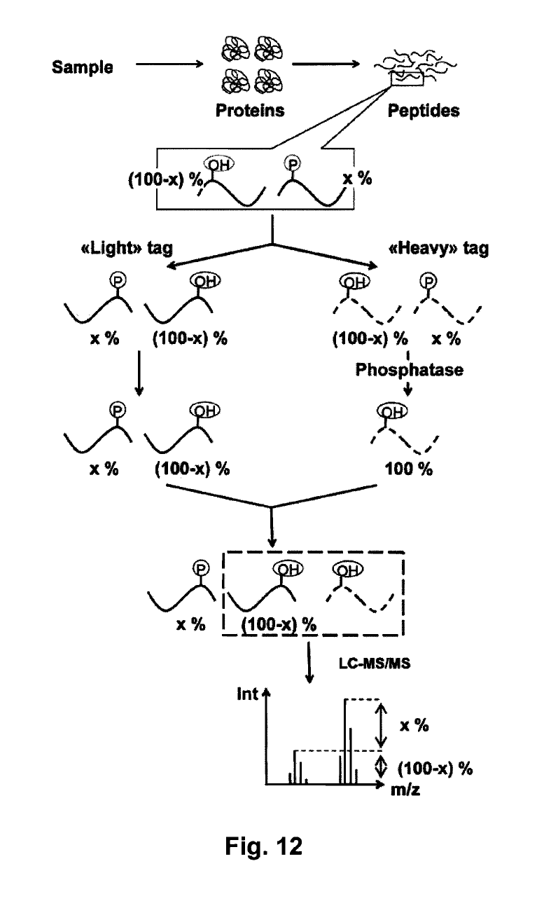

Before analyzing the proteins from isolated LDs, the efficiency and precision of the method was tested using purified bovine α-casein. Using a 500 fmol aliquot of total protein all previously-reported phosphorylated peptides could be detected (Table III). Accuracy of quantifications was evaluated using 11 mixtures containing native and enzymatically dephosphorylated α-casein in ratios from 1:9 to 9:1. When the samples were analyzed and compared by isotope labelling and LC-MS/MS, we found that in this range of concentrations the relative protein abundance was measured with high accuracy (FIG. 13).

TABLE III
Phosphorylated peptides detected in bovine α-casein.

| | SEQ | Reported | | |
|---|---|---|---|---|
| | ID | phosphorylation | Isotope | Standard |
| Peptide sequence | NO: | site | ratio | deviation |
| DIGS*ES*TEDQAMEDIK | 3 | S61/63 | 8.3 | 0.6 |
| QMEAES*IS*S*S*EEIVPNSVEQK | 42 | S79/81/82/83 | 8.6 | 0.6 |
| TVDMES*TEVFTK | 5 | S158 | 7.0 | 0.5 |
| NMAINPS*K | 43 | S46 | 6.6 | 0.4 |
| YKVPQLEIVPNS*AEER | 8 | S130 | 6.4 | 0.3 |

Reported phosphorylation sites are marked with asterisks. Data show mean values and standard deviations of 3 independent experiments.

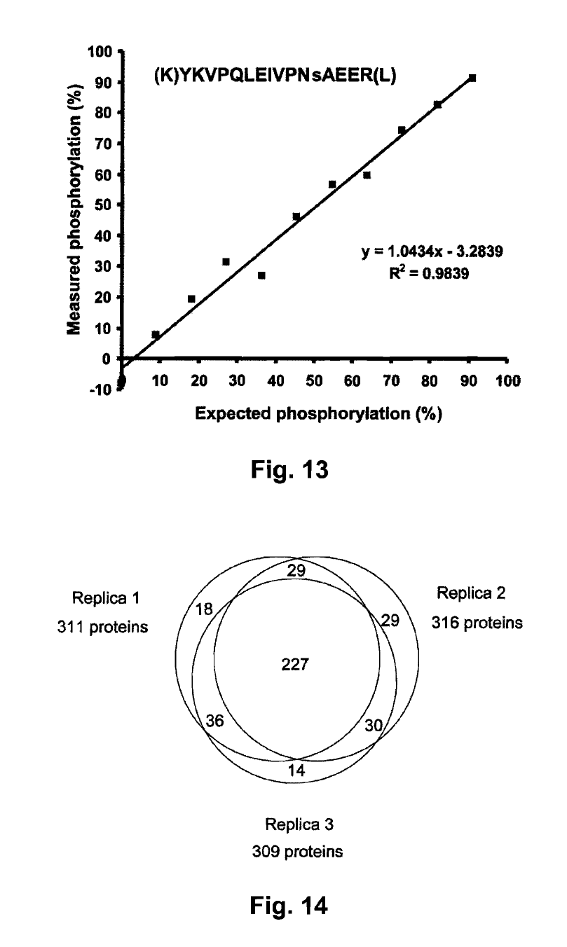

Three biological replicas were analyzed for each state. The light and heavy labels were alternated between replicas in order to eliminate any possible isotope-specific effects in the analysis. In total ˜52,500 MS/MS spectra were collected in three replicas, leading to detection of 3105 non-redundant peptides (FDR <0.05); 384 proteins were identified by at least 2 unique peptides (FIG. 14 and Table IV). ˜70% of peptides were labeled with SIMPL tags in each replica.

TABLE IV
Proteins identified in purified lipid droplets from mouse WAT.

| | | Number | | |
|---|---|---|---|---|
| Accession | | of | | |
| number | Protein name | peptides | Score | Coverage, % |
| P19096 | Fatty acid synthase | 55 | 849.45 | 31 |
| O88492 | Adipocyte protein S3-12 | 34 | 605.62 | 36 |
| A3KGU5 | Alpha-II spectrin | 42 | 592.45 | 26 |
| P20152 | Vimentin | 32 | 534.68 | 56 |
| Q8CGN5 | Perilipin | 25 | 440.32 | 53 |
| A0JLR7 | AHNAK | 29 | 402.79 | 18 |
| Q3UHH3 | Perlecan | 28 | 377.79 | 11 |
| Q6XD99 | Non-erythroid spectrin beta | 24 | 340.5 | 16 |
| Q63862 | Myosin-11 | 23 | 326.11 | 17 |
| Q3T9S7 | Pyruvate carboxylase | 20 | 321.78 | 24 |
| Q9Z0I9 | Collagen VI, alpha-3 | 22 | 317.95 | 12 |
| Q3TEK2 | Heat shock protein 8 | 19 | 310.27 | 34 |
| A2AQ53 | Fibrillin | 23 | 297.23 | 14 |
| Q921I1 | Serotransferrin | 21 | 296.95 | 29 |
| A2ASS6 | Titin | 31 | 290.94 | 1 |
| Q3UNG0 | Laminin, gamma-1 | 19 | 288.92 | 18 |
| Q8BX70 | Vacuolar protein sorting protein 13C | 19 | 276.03 | 8 |
| Q3TPN0 | Nidogen-2 | 17 | 271.01 | 22 |
| P16015 | Carbonic anhydrase 3 | 16 | 265.18 | 74 |
| Q6GTG6 | Very long chain Acyl-CoA synthetase 1 | 17 | 253.04 | 33 |
| A2AIM8 | Talin | 20 | 228.53 | 14 |
| O08590 | Semicarbazide-sensitive amine oxidase/vascular | 12 | 222.31 | 24 |
| | adhesion protein-1 | | | |
| Q71LX8 | HSP90-beta | 13 | 221.62 | 25 |
| A2AM91 | Filamin A | 17 | 211.52 | 11 |
| Q5SWU9 | Acetyl-CoA carboxylase 1 | 16 | 209.4 | 9 |
| Q03265 | Mitochondrial ATP synthase subunit alpha | 12 | 201.79 | 30 |
| O54724 | Polymerase I and transcript release factor (PTRF) | 12 | 200.82 | 41 |
| Q3TVM2 | Aldehyde dehydrogenase 2 | 12 | 200.47 | 34 |
| P56480 | Mitochondrial ATP synthase, subunit beta | 12 | 199.24 | 32 |
| Q8BH64 | EH domain-containing protein 2 | 13 | 196.94 | 36 |
| Q07936 | Annexin A2 | 12 | 195.35 | 41 |
| Q3TBQ4 | Myosin IC | 15 | 188.07 | 18 |
| Q3TUI1 | Annexin A6 | 12 | 187.42 | 29 |
| P54310 | Lipase, hormone sensitive | 12 | 186.31 | 20 |
| Q8R431 | Monoglyceride lipase | 12 | 185.32 | 41 |
| Q14BF2 | Laminin, alpha-4 | 13 | 169.81 | 13 |
| Q3TKX9 | Nidogen-1 | 13 | 164.17 | 15 |
| Q8BFZ3 | Kappa actin | 9 | 163.6 | 40 |
| Q3TIZ0 | Tubulin, alpha-6 | 10 | 163.04 | 34 |
| Q3U733 | Lamin A | 12 | 157.65 | 26 |
| Q3KQQ4 | Serpin 1a | 10 | 156.88 | 26 |
| P07759 | Contrapsin | 9 | 150.9 | 24 |
| Q3TW93 | Heat shock protein A | 9 | 148.61 | 19 |
| Q99KI0 | Aconitase | 11 | 145.94 | 24 |
| Q3USI2 | Laminin, beta-2 | 11 | 144.16 | 10 |
| Q3UHL7 | Laminin, beta-1, subunit 1 | 10 | 143.56 | 9 |
| Q8VCT4 | Triacylglycerol hydrolase | 9 | 138.39 | 23 |
| Q549D9 | Beta-globin | 8 | 138.25 | 60 |
| Q64727 | Vinculin | 10 | 138.09 | 16 |
| Q9CZ13 | Ubiquinol-cytochrome-c reductase complex core | 10 | 134.24 | 33 |
| | protein 1, mitochondrial | | | |
| Q3U9V4 | Guanine nucleotide binding protein, beta 2 | 9 | 134.23 | 36 |
| Q3TJ98 | Clathrin, heavy polypeptide | 9 | 133.13 | 9 |
| Q4QRB4 | Tubulin, beta-3 | 9 | 132.86 | 37 |
| Q6S385 | Plectin 10 | 14 | 132.68 | 6 |
| Q4FJQ0 | Rab 7 | 9 | 132.27 | 46 |
| Q80U36 | Dynein, cytoplasmic 1 | 11 | 131.4 | 3 |
| Q3TJK3 | HSP47 | 8 | 130.33 | 22 |
| Q5FW97 | Enolase | 8 | 128.93 | 30 |
| Q3TXK7 | G protein alpha(i2) (GNAI2) | 8 | 127.66 | 30 |
| Q05DV1 | Por | 9 | 127.11 | 20 |
| Q542H7 | Fatty acid binding protein 4/fatty-acid-binding protein | 8 | 127.08 | 45 |
| | aP2 | | | |
| Q3UA58 | 14-3-3 zeta | 8 | 127.02 | 43 |
| Q8BNF8 | Valosin containing protein | 8 | 125.57 | 18 |
| P27773 | ERp57/GRP58 | 9 | 124.21 | 25 |
| Q61753 | 3-Phosphoglycerate dehydrogenase | 9 | 120.24 | 23 |
| Q9DB77 | Ubiquinol-cytochrome-c reductase complex core | 7 | 119.13 | 23 |
| | protein 2, mitochondrial | | | |
| Q8R2Y2 | MUC-18, CD146 | 8 | 118.8 | 18 |
| A2AQX3 | Putative strictosidine synthase | 8 | 115.51 | 24 |
| Q63918 | Serum deprivation response protein | 8 | 114.59 | 37 |
| O08855 | Apolipoprotein A-I | 7 | 114.27 | 24 |
| Q3TUD6 | HSP90B1 | 8 | 113.05 | 11 |
| P26443 | Glutamate dehydrogenase 1 | 8 | 112.39 | 20 |
| A2AL35 | Gelsolin | 8 | 111.57 | 17 |
| Q3TLV2 | Transglutaminase 2 | 8 | 110.41 | 19 |
| A2AH73 | 6-Phosphogluconate dehydrogenase | 7 | 110.4 | 19 |
| Q02788 | Collagen VI, alpha-2 | 7 | 109.7 | 9 |
| A2A4H2 | ATP citrate lyase | 10 | 109.35 | 15 |
| Q3TBB4 | Fibronectin 1. | 9 | 109.12 | 7 |
| Q6P7S0 | Pyruvate kinase | 8 | 108.42 | 27 |
| Q5GQ64 | Gamma-synuclein | 6 | 108.11 | 61 |
| P08249 | Malate dehydrogenase | 7 | 107.32 | 29 |
| Q549A5 | Clusterin | 8 | 107.13 | 23 |
| Q3TXF9 | Na(+)/K(+) ATPase | 8 | 106.55 | 10 |
| Q3TVZ1 | Catalase | 7 | 100.62 | 17 |
| A2AM44 | Ubiquitin-activating enzyme E1, Chr X | 7 | 100.57 | 12 |
| Q3TDA2 | Methylmalonate-semialdehyde dehydrogenase | 7 | 99.62 | 22 |
| Q99K47 | Fibrinogen, alpha-chain | 7 | 98.57 | 24 |
| A6ZI44 | Aldolase A | 6 | 96.03 | 13 |
| Q3TA14 | CD36 | 5 | 95.22 | 19 |
| Q3UNQ6 | Erythrocyte protein band 4.1-like 2 | 7 | 94.38 | 14 |
| Q3U7R1 | Extended synaptotagmin-1 | 7 | 94.35 | 10 |
| Q32MX7 | Kng1 | 6 | 93.63 | 14 |
| Q0PD66 | Rab1B | 6 | 92.1 | 47 |
| Q9DBS1 | Transmembrane protein 43 | 6 | 91.88 | 25 |
| Q3TVD2 | Calreticulin | 6 | 89.37 | 21 |
| Q1A602 | Alpha-actinin-4 | 7 | 88.91 | 11 |
| O08614 | Utrophin | 8 | 88.52 | 4 |
| P63038 | HSP60 | 6 | 88.06 | 18 |
| Q61838 | Alpha-2-macroglobulin | 7 | 87.78 | 7 |
| Q3U5N9 | Annexin 1/lipocortin 1 | 7 | 87.71 | 32 |
| P13707 | Glycerol-3-phosphate dehydrogenase 1 | 6 | 87.69 | 22 |
| Q923B6 | STAMP2 | 6 | 87.4 | 18 |
| P69897 | Tubulin, beta-5 | 4 | 87.11 | 8 |
| P62804 | Histone H4 | 6 | 86.21 | 44 |
| Q66HF1 | NADH-ubiquinone oxidoreductase 75 kDa subunit, | 7 | 84.58 | 16 |
| | mitochondrial | | | |
| Q61287 | Alpha-globin | 5 | 84.31 | 49 |
| Q9CY59 | Cytochrome P450 oxidoreductase | 5 | 83.93 | 22 |
| Q99LW4 | Collagen VI, alpha-1 | 6 | 83.91 | 11 |
| P40142 | Transketolase | 6 | 83.83 | 20 |
| Q3TFP8 | progesterone receptor membrane component 1, | 6 | 83.26 | 49 |
| | PGRMC1/HPR6.6 | | | |
| A0JLV3 | Histone H2B | 6 | 82.94 | 41 |
| Q3UKP2 | Hemopexin | 6 | 82.84 | 22 |
| P08122 | Collagen IV, alpha-2 | 6 | 81.74 | 7 |
| P11507 | Sarcoplasmic/endoplasmic reticulum calcium | 6 | 81.66 | 9 |
| | ATPase 2 | | | |
| A2A8X4 | ATP synthase, H+ transporting, mitochondrial F0 | 5 | 81.59 | 42 |
| | complex, subunit d | | | |
| Q3TCI7 | Lactate dehydrogenase A | 6 | 81.48 | 22 |
| Q3TII3 | Elongation factor 1-alpha | 5 | 81.17 | 23 |
| Q3TU16 | ABCD2 (ALDR) transporter | 6 | 81.15 | 11 |
| Q3TT11 | D-3-hydroxyacyl-CoA dehydratase/D-3-hydroxyacyl- | 5 | 80.7 | 10 |
| | CoA dehydrogenase bifunctional protein | | | |
| Q5U5Y5 | Long chain 3-hydroxyacyl-CoA dehydrogenase | 6 | 80.46 | 14 |
| A2AP14 | Peroxiredoxin 1 | 6 | 80.29 | 40 |
| A2A7C0 | Microtubule-actin crosslinking factor 1 | 8 | 80 | 2 |
| Q3UKW2 | Calmodulin 1 | 5 | 78.66 | 46 |
| Q3UJR6 | Very-long-chain acyl-coenzyme A dehydrogenase | 7 | 77.6 | 20 |
| P48036 | Annexin A5 | 5 | 76.98 | 24 |
| Q3TED2 | Programmed cell death 6 interacting protein | 7 | 76 | 16 |
| P00173 | Cytochrome B5 | 4 | 75.96 | 41 |
| A2VCW2 | Caveolin-1 | 4 | 74.56 | 28 |
| Q3UCL7 | Ribosomal protein S3 | 6 | 74.43 | 31 |
| Q6AYS2 | Sideroflexin 1 | 5 | 74.35 | 17 |
| Q3UPA1 | guanine nucleotide binding protein, alpha 11 | 4 | 73.93 | 14 |
| Q9DBG5 | TIP47 (Mannose-6-phosphate receptor-binding | 6 | 73.67 | 28 |
| | protein 1) | | | |
| Q91YH5 | Atlastin-3 | 6 | 72.36 | 23 |
| A2ACM8 | 14-3-3-epsilon | 5 | 72.31 | 27 |
| Q9JJA0 | Glutation transferase zeta | 6 | 71.93 | 35 |
| P61982 | 14-3-3-gamma | 4 | 71.59 | 25 |
| Q3TQP6 | Malic enzyme, cytosolic | 5 | 71.41 | 16 |
| Q3UGC8 | Propionyl-Coenzyme A carboxylase, alpha | 6 | 71.35 | 14 |
| | polypeptide | | | |
| Q3TJZ1 | Eukaryotic translation elongation factor 2 | 6 | 71.24 | 13 |
| Q3TGR2 | Fibrinogen B, beta-chain | 5 | 70.62 | 14 |
| Q3UFR4 | Neutral amino acid transporter ASCT2 | 4 | 69.89 | 11 |
| Q5EB77 | Rab18 | 4 | 69.05 | 30 |
| Q3U7H9 | Peroxiredoxin 5. | 6 | 68.6 | 40 |
| Q3TIT9 | 3-Oxoacyl-CoA thiolase | 6 | 68.48 | 23 |
| Q542P4 | MPP1 | 5 | 68.26 | 16 |
| P15105 | Glutamine synthetase | 4 | 67.77 | 14 |
| Q3U900 | Ribophorin I | 5 | 67.71 | 12 |
| P14152 | Malate dehydrogenase, cytoplasmic | 5 | 67.66 | 23 |
| Q63258 | Integrin alpha-7 | 6 | 67.33 | 10 |
| Q3UL78 | Cell division cycle 42 homolog | 4 | 67.02 | 25 |
| P51174 | Long-chain specific acyl-CoA dehydrogenase, | 5 | 66.83 | 15 |
| | mitochondrial | | | |
| Q3TH64 | Cytochrome b5, type B | 4 | 66.26 | 50 |
| Q60932 | Voltage-dependent anion channel protein 1, VDAC1 | 5 | 66.04 | 30 |
| B1B1B4 | Electron transferring flavoprotein, alpha polypeptide | 4 | 65.85 | 11 |
| P45592 | Cofilin-1 | 5 | 65.51 | 40 |
| Q3UFJ3 | Pyruvate dehydrogenase E1 alpha-subunit | 5 | 65.27 | 18 |
| Q60597 | 2-Oxoglutarate dehydrogenase E1 component, | 5 | 64.95 | 8 |
| | mitochondrial | | | |
| Q3TIC2 | Rhob | 3 | 64.49 | 26 |
| Q6IRU2 | Tropomyosin 4, alpha | 4 | 64.08 | 24 |
| A6H6A8 | Glyceraldehyde 3-phosphate dehydrogenase | 2 | 64 | 6 |
| Q5QGU0 | Receptor expression enhancing protein 5, REEP5 | 5 | 63.84 | 21 |
| Q3TNH0 | Thymopoietin | 5 | 63.83 | 20 |
| A6H584 | Collagen VI, alpha-5 | 4 | 63.73 | 3 |
| Q61171 | Peroxiredoxin-2 | 4 | 63.44 | 22 |
| Q5XI73 | Rho GDP dissociation inhibitor alpha | 4 | 62.95 | 20 |
| Q0PGA1 | Cytochrome P450 2E1 | 4 | 62.54 | 10 |
| Q0QF18 | Succinate dehydrogenase complex subunit A | 5 | 62.44 | 17 |
| Q80XP1 | Complement component 3 | 6 | 62.43 | 5 |
| Q3TF72 | Prolyl 4-hydroxylase, beta polypeptide | 4 | 62.13 | 12 |
| Q3TX45 | Apolipoprotein E | 4 | 62.09 | 18 |
| Q3TVD9 | Methylcrotonoyl-Coenzyme A carboxylase 1 | 5 | 61.81 | 11 |
| A2ACK5 | Solute carrier family 25 | 5 | 61.77 | 29 |
| Q642K0 | Muscle myosin alkali light chain 6 | 4 | 61.19 | 25 |
| Q3UQ71 | Epoxide hydrolase 2, cytoplasmic | 4 | 61.07 | 13 |
| P51885 | Lumican | 4 | 60.17 | 20 |
| Q3U0Q9 | Tensin 1 | 4 | 60.08 | 7 |
| Q3TN26 | Fatty acid transport protein-1, FATP-1 | 5 | 59.75 | 14 |
| Q3U1N3 | R-Ras | 4 | 59.62 | 29 |
| Q3TSV1 | Decorin | 4 | 59.44 | 20 |
| P42208 | Nedd5/Septin-2 | 4 | 57.97 | 21 |
| Q3TIU3 | alpha-2-HS-glycoprotein (fetuin) | 4 | 57.9 | 20 |
| Q6PD20 | Mccc2 protein | 5 | 57.48 | 15 |
| P19783 | Cytochrome c oxidase subunit 4 isoform 1, | 4 | 57.43 | 30 |
| | mitochondrial | | | |
| Q52JJ6 | Glutamyl aminopeptidase/aminopeptidase A | 4 | 57.4 | 6 |
| Q3UA25 | Ribosomal protein S8. | 4 | 57.05 | 24 |
| Q3TC17 | Cathepsin B | 4 | 56.82 | 14 |
| Q545A2 | ADP/ATP translocase | 3 | 56.79 | 17 |
| A3KGV9 | Outer dense fiber of sperm tails 2 | 4 | 56.66 | 7 |
| P07871 | 3-Ketoacyl-CoA thiolase B, peroxisomal | 4 | 56.45 | 18 |
| Q8R0Y6 | 10-Formyltetrahydrofolate dehydrogenase | 5 | 56.21 | 8 |
| Q8QZS1 | 3-Hydroxyisobutyryl-CoA hydrolase, mitochondrial | 4 | 56.16 | 15 |
| P16045 | Galectin-1 | 3 | 55.9 | 31 |
| Q60662 | A-kinase anchor protein 4 | 5 | 55.87 | 9 |
| Q50HX1 | RAB14 protein variant | 3 | 55.8 | 25 |
| Q6SA19 | Triosephosphate isomerase | 4 | 55.7 | 23 |
| Q0PD65 | Rab2 | 3 | 55.01 | 20 |
| P17563 | Selenium-binding protein 1 | 4 | 54.9 | 16 |
| Q3TIQ2 | Ribosomal protein L12 | 4 | 54.88 | 40 |
| Q3TAF9 | Biglycan | 4 | 54.44 | 15 |
| Q3T9A1 | EH-domain containing protein 1. | 4 | 53.08 | 11 |
| Q8C355 | Cu/Zn superoxide dismutase | 3 | 52.67 | 25 |
| Q68FG5 | Eukaryotic translation elongation factor 1 delta | 4 | 52.63 | 31 |
| Q62087 | Serum paraoxonase/lactonase 3 | 4 | 51.95 | 17 |
| Q8BH78 | NOGO | 5 | 51.84 | 7 |
| Q0PD45 | Rab11A | 3 | 51.57 | 19 |
| Q3THH1 | Endoplasmic reticulum protein 5, ERp5/PDIA6/P5 | 3 | 51.55 | 11 |
| A2A895 | VAMP 3 | 3 | 51.44 | 38 |
| Q9DCW4 | Electron transfer flavoprotein subunit beta | 3 | 51.38 | 19 |
| Q3U7Z6 | Phosphoglycerate mutase 1 | 4 | 51.09 | 18 |
| Q9D0M3 | Cytochrome c1 heme protein, mitochondrial | 4 | 50.99 | 21 |
| Q497I3 | Fatty acid binding protein 5, epidermal | 4 | 50.14 | 34 |
| P06728 | Apolipoprotein A-IV | 3 | 50.13 | 9 |
| Q5M9J9 | Aldo-keto reductase 1B7 | 3 | 49.98 | 16 |
| Q9CR68 | Ubiquinol-cytochrome c reductase iron-sulfur | 4 | 49.63 | 20 |
| | subunit, mitochondrial | | | |
| Q3TQP7 | Acetoacetyl-CoA thiolase | 4 | 49.5 | 17 |
| Q8QZV3 | Dodecenoyl-Coenzyme A delta isomerase | 3 | 49.35 | 15 |
| A2CEK3 | Phosphoglucomutase 2 | 4 | 49.18 | 12 |
| Q60930 | Voltage-dependent anion channel protein 2, VDAC2 | 4 | 48.89 | 18 |
| Q3TJ53 | Tropomyosin 3, gamma | 3 | 48.5 | 10 |
| Q3TIW5 | Integrin beta 1 | 4 | 48.49 | 7 |
| P33622 | Apolipoprotein C-III | 3 | 47.53 | 42 |
| Q3TJ39 | Rab-5C | 4 | 47.49 | 31 |
| Q3UGX2 | Beta-I spectrin | 5 | 46.92 | 3 |
| A2AE89 | Glutathione S-transferase M1 | 3 | 46.6 | 12 |
| Q8C064 | Protein kinase C delta-binding protein | 3 | 46.52 | 32 |
| Q641Y2 | NADH dehydrogenase (complex1) iron-sulphur | 4 | 46.01 | 14 |
| | protein 2 | | | |
| P39061 | Collagen XVIII alpha-1 | 4 | 45.98 | 7 |
| A2RT07 | Reticulocalbin 2 | 4 | 45.81 | 23 |
| Q922Z5 | CGI-58/ABHD5 | 3 | 45.53 | 15 |
| Q5NC82 | Nucleoside diphosphate kinase | 3 | 45.52 | 26 |
| Q9JK53 | Prolargin | 3 | 45.42 | 11 |
| Q9D6R2 | NAD(+)-specific isocitrate dehydrogenase alpha- | 3 | 45.35 | 12 |
| | subunit | | | |
| Q3TXE5 | Calnexin | 3 | 45.16 | 7 |
| Q3ULL5 | Eukaryotic translation initiation factor 2, subunit 2 | 4 | 45.1 | 18 |
| Q3U6G1 | Biliverdin reductase B | 4 | 44.54 | 24 |
| Q922Q1 | MOSC domain-containing protein 2, mitochondrial | 3 | 43.96 | 11 |
| Q3ULV8 | Diazepam binding inhibitor | 3 | 43.86 | 50 |
| Q9R257 | Heme-binding protein 1 | 3 | 42.83 | 26 |
| P97450 | ATP synthase-coupling factor 6, mitochondrial | 3 | 42.78 | 34 |
| P50399 | Rab GDP dissociation inhibitor beta | 3 | 42.45 | 11 |
| O55107 | Basigin | 3 | 42.4 | 13 |
| P58871 | TAB182 | 3 | 42.3 | 4 |
| Q9D8W6 | Adenylate kinase 3 alpha like | 3 | 42.18 | 20 |
| A2ACG6 | Ribophorin II | 3 | 41.99 | 7 |
| Q544G5 | Dystroglycan 1 | 3 | 41.51 | 5 |
| Q76MZ3 | Serine/threonine-protein phosphatase 2A 65 kDa | 3 | 41.25 | 8 |
| | regulatory subunit A alpha isoform | | | |
| Q3T9Z2 | Glyoxylate reductase/hydroxypyruvate reductase | 2 | 41 | 6 |
| Q9Z2A7 | Diacylglycerol O-acyltransferase 1 | 3 | 40.99 | 12 |
| P21614 | Vitamin D-binding protein | 3 | 40.83 | 14 |
| Q9D6J6 | NADH dehydrogenase [ubiquinone] flavoprotein 2, | 2 | 40.82 | 8 |
| | mitochondrial | | | |
| Q3U3C3 | Lipin 1 | 3 | 40.33 | 7 |
| Q3U944 | Lectin, mannose-binding, 1 | 3 | 39.67 | 11 |
| P14733 | Lamin-B1 | 3 | 39.63 | 7 |
| Q6XFR5 | Glycophorin C | 2 | 39.56 | 28 |
| Q3UNI8 | D-Dopachrome tautomerase | 3 | 39.44 | 42 |
| Q924U3 | Caveolin-2 | 3 | 38.9 | 33 |
| A2AU04 | Integrin, alpha-6 | 3 | 38.81 | 7 |
| Q3U7U8 | Rap1a | 3 | 38.68 | 23 |
| Q3TIE8 | Dihydrolipoyl dehydrogenase | 3 | 38.52 | 8 |
| Q8BJ56 | Adipose triglyceride lipase | 3 | 38.18 | 14 |
| Q3TX25 | Aldehyde dehydrogenase 7 | 2 | 38.17 | 5 |
| Q8BRF7 | MUNC18-1/Syntaxin-binding protein 1 | 2 | 37.96 | 5 |
| Q499V7 | GTP-specific succinyl-CoA synthetase | 3 | 37.94 | 9 |
| Q8K4K8 | Antioxidant protein | 2 | 37.9 | 4 |
| Q3UF03 | Mannose-6-phosphate receptor, cation dependent | 3 | 37.71 | 14 |
| A2AP21 | Aldehyde dehydrogenase 4 family, member A1 | 2 | 37.64 | 2 |
| Q3TGZ3 | Isocitrate dehydrogenase 3, gamma | 2 | 37.48 | 11 |
| P60335 | Poly(rC)-binding protein 1 | 2 | 37.26 | 7 |
| A2ALM8 | Beta-cell receptor-associated protein 31 (BAP 31) | 3 | 36.92 | 12 |
| Q6ZQI3 | Uncharacterized protein KIAA0152 | 3 | 36.88 | 16 |
| A5JUZ1 | Ubiqutin subunit 1 | 2 | 36.67 | 23 |
| Q91XV3 | Neuronal axonal membrane protein NAP-22 | 3 | 36.65 | 32 |
| Q3TNR7 | BCL2-like 13 | 3 | 36.47 | 14 |
| Q9CXR1 | Dehydrogenase/reductase SDR family member 7 | 3 | 36.09 | 14 |
| Q3U4U1 | Periostin | 3 | 36 | 6 |
| Q3UZ06 | SEC22 vesicle trafficking protein-like 1 | 3 | 35.94 | 27 |
| Q5NCJ9 | Ubiquinol-cytochrome C reductase complex 7.2 kDa | 2 | 35.6 | 26 |
| | protein | | | |
| A1L0X5 | Keratin 5B | 3 | 35.57 | 8 |
| P84099 | 60S ribosomal protein L19 | 2 | 35.24 | 8 |
| A2A7Z6 | Rab-3B | 2 | 35.15 | 8 |
| Q3ULN8 | protein phosphatase 2, regulatory subunit B | 3 | 35.1 | 8 |
| P00920 | Carbonic anhydrase 2 | 3 | 35.04 | 24 |
| Q3TCL2 | Aldo-keto reductase 1B3 | 3 | 34.99 | 13 |
| Q3TRX4 | Paralemmin | 2 | 34.45 | 9 |
| Q3UKX6 | Apolipoprotein A-II | 2 | 34.44 | 32 |
| Q8C6E9 | Purine rich element binding protein A | 2 | 34.32 | 15 |
| Q3TEE8 | Thioredoxin domain containing 5 | 3 | 34.29 | 13 |
| Q9CR61 | NADH ubiquinone dehydrogenase 1 beta | 2 | 34.11 | 21 |
| | subcomplex subunit 7 | | | |
| A2A820 | Adenylate kinase 2 | 3 | 34.04 | 15 |
| P05784 | Keratin 18 | 2 | 33.98 | 9 |
| Q7TNQ6 | Ribosomal phosphoprotein P2 | 2 | 33.92 | 42 |
| Q3TL33 | Calumenin | 2 | 33.89 | 12 |
| Q6ZQF0 | DNA topoisomerase II-binding protein 1 | 3 | 33.81 | 3 |
| A2AT05 | Peptidyl-prolyl cis-trans isomerase | 2 | 33.68 | 12 |
| Q5RJH8 | Glutathione peroxidase 1 | 2 | 33.64 | 9 |
| A0JNU4 | Carbamoyl-phosphate synthetase 1 | 3 | 33.47 | 2 |
| A2A5D1 | Signal transducer and activator of transcription 3 | 2 | 33.42 | 3 |
| Q8BJL4 | VIP 36 | 3 | 33.18 | 6 |
| Q9WV55 | Vesicle-associated membrane protein-associated | 3 | 33.03 | 18 |
| | protein A/VAP33 | | | |
| A2A9Z0 | Dystrophin, muscular dystrophy | 3 | 33.01 | 1 |
| A6H589 | Collagen VI alpha-6 | 2 | 32.87 | 1675 |
| P13541 | Myosin-3 | 3 | 32.82 | 3 |
| Q80TQ1 | Phosphatidylinositide phosphatase SAC1 | 3 | 32.72 | 8 |
| A2AKX3 | Senataxin | 3 | 32.7 | 2 |
| Q0QER9 | Isocitrate dehydrogenase 1 | 3 | 32.08 | 8 |
| A6H5X0 | 1-Acylglycerol-3-phosphate O-acyltransferase 9 | 2 | 32.05 | 4 |
| Q05DE0 | ADP-ribosylation factor GTPase activating protein 1 | 2 | 31.99 | 8 |
| P11404 | Fatty acid-binding protein, heart | 2 | 31.96 | 6 |
| Q8CJG5 | Afadin | 2 | 31.93 | 9 |
| Q9JLC8 | Sacsin | 3 | 31.84 | 1 |
| Q53ZD4 | Microsomal glutathione S-transferase 1 | 2 | 31.47 | 11 |
| P09411 | Phosphoglycerate kinase 1 | 2 | 31.32 | 10 |
| Q6GTX4 | Adiponectin | 2 | 31.25 | 10 |
| Q8BK95 | Myosin VI homolog | 2 | 31.08 | 1 |
| Q9CQ75 | NADH dehydrogenase [ubiquinone] 1 alpha | 2 | 31.04 | 14 |
| | subcomplex subunit 2 | | | |
| Q3T9U8 | Ectonucleoside triphosphate diphosphohydrolase 2 | 2 | 30.79 | 5 |
| Q9D881 | Cytochrome c oxidase, subunit Vb | 3 | 30.67 | 14 |
| Q3UNN1 | Septin 7 | 3 | 30.65 | 10 |
| A2AQP5 | Thioredoxin domain containing 4 | 3 | 30.34 | 14 |
| Q4FJK0 | 2,4-Dienoyl-CoA reductase | 2 | 30.26 | 8 |
| P02466 | Collagen I alpha-2 | 3 | 30.12 | 5 |
| A2ACH6 | Delta-sarcoglycan | 2 | 29.66 | 15 |
| A2AAW9 | Eukaryotic translation initiation factor 2, subunit 3, | 3 | 29.61 | 11 |
| | structural gene X-linked | | | |
| Q5I0T9 | Glycine N-methyltransferase | 2 | 29.5 | 6 |
| Q5BLJ9 | 60S ribosomal protein L27 | 2 | 29.38 | 15 |
| P70412 | CUB and zona pellucida-like domain-containing | 2 | 28.75 | 6 |
| | protein 1 | | | |
| A2APS2 | Sterol carrier protein 2 | 2 | 28.61 | 19 |
| Q3TAJ4 | Band 3 protein | 2 | 28.51 | 3 |
| Q3TJN3 | Cathepsin D | 3 | 28.49 | 12 |
| Q3U8W4 | I/Mn superoxide dismutase | 2 | 28.38 | 13 |
| Q3TF84 | Leucine rich repeat containing 59 | 2 | 28.35 | 16 |
| Q3V2I9 | Acetyl-Coenzyme A dehydrogenase, medium chain. | 2 | 28.1 | 8 |
| Q5M9L7 | Ribosomal protein S17 | 2 | 27.85 | 17 |
| A2A850 | Acyl-Coenzyme A oxidase 1, palmitoyl | 2 | 27.71 | 5 |
| Q9CPQ8 | ATP synthase subunit g, mitochondrial | 2 | 27.36 | 18 |
| Q7TMG7 | Natriuretic peptide receptor C | 2 | 27.2 | 4 |
| Q8BMF4 | Dihydrolipoyllysine-residue acetyltransferase | 2 | 27.19 | 4 |
| | component of pyruvate dehydrogenase complex, | | | |
| | mitochondrial | | | |
| Q8C4T0 | Retinoic acid induced 16 | 2 | 27.12 | 7 |
| Q8CA90 | H2A histone family, member Y | 2 | 27.1 | 10 |
| Q3TW65 | Ribosomal protein S18 | 2 | 27 | 13 |
| Q99JY0 | 3-ketoacyl-CoA thiolase, beta subunit | 2 | 26.84 | 4 |
| Q3UP74 | Alanyl aminopeptidase | 3 | 26.77 | 5 |
| Q3TVJ8 | TRAP-delta | 2 | 26.43 | 16 |
| A2A6T7 | ADP-ribosylation factor 2 | 2 | 26.32 | 17 |
| Q3US68 | Inter-alpha-trypsin inhibitor heavy chain H5 | 2 | 26.13 | 3 |
| Q8R149 | BUD13 homolog | 2 | 25.78 | 8 |
| P70206 | Plexin-A1 | 2 | 25.76 | 0 |
| Q543J4 | S14 protein | 2 | 25.63 | 23 |
| A6X957 | ER-associated dnaJ protein 3 | 2 | 25.59 | 5 |
| Q9CQB4 | Ubiquinol-cytochrome C reductase binding protein | 2 | 25.29 | 20 |
| Q3UIA9 | Fumarate hydratase 1 | 2 | 25.04 | 6 |
| Q91VG6 | Peptidyl-prolyl cis-trans isomerase B | 2 | 25.03 | 14 |
| A2BDX1 | Dolichol-phosphate (Beta-D) mannosyltransferase 1 | 2 | 24.99 | 9 |
| Q9D2W1 | Cytochrome c oxidase, subunit Va | 2 | 24.86 | 10 |
| Q3TGW2 | Endonuclease/exonuclease/phosphatase family | 2 | 24.8 | 7 |
| | domain-containing protein 1 | | | |
| Q4V9X9 | Ribosomal protein L23a | 2 | 24.7 | 8 |
| Q3UA32 | UDP-N-acetyl-alpha-D-galactosamine:polypeptide N- | 2 | 24.66 | 6 |
| | acetylgalactosaminyltransferase 2. | | | |
| Q547K2 | Solute carrier family 22, member 3 | 2 | 24.61 | 4 |
| Q8BH95 | Enoyl-CoA hydratase, mitochondrial | 2 | 24.59 | 15 |
| A2A909 | Ubiquinol-cytochrome c reductase hinge protein | 2 | 24.49 | 30 |
| Q3TII0 | Chaperonin containing TCP-delta | 2 | 24.16 | 5 |
| Q9D1L9 | Hepatitis B virus X-interacting protein homolog | 2 | 24.1 | 21 |
| Q3TVX7 | Synaptophysin-like protein | 2 | 23.76 | 13 |
| Q9QZC4 | Choline-phosphate cytidylyltransferase B | 2 | 23.14 | 5 |
| Q91WS0 | CDGSH iron sulfur domain-containing protein 1 | 2 | 23.08 | 25 |
| | (mitoNEET) | | | |
| Q3UC51 | Oligosaccharyltransferase | 2 | 22.98 | 3 |
| Q63ZW9 | Cop A | 2 | 22.9 | 2 |
| Q52KH6 | Hypothetical protein LOC574404 | 2 | 22.5 | 1 |
| P52503 | NADH dehydrogenase [ubiquinone] iron-sulfur | 2 | 21.99 | 33 |
| | protein 6, mitochondrial | | | |
| P70459 | ETS domain-containing transcription factor ERF | 2 | 21.9 | 8 |
| Q6PF96 | Electron transferring flavoprotein, dehydrogenase | 2 | 21.86 | 6 |
| A2AMX4 | Synapse associated protein 1 | 2 | 21.69 | 5 |
| Q8BG11 | Cadherin 13 | 2 | 21.67 | 3 |
| O08709 | Peroxiredoxin-6 | 2 | 21.63 | 14 |
| Q8BN84 | hypothetical Trp-Asp | 2 | 21.48 | 2 |
| Q7TSI1 | Adaptor protein 162 | 2 | 21.42 | 2 |
| Q3UF75 | Parvin, alpha | 2 | 21.06 | 8 |
| P24472 | Glutathione S-transferase A4 | 2 | 20.61 | 16 |
| Q80WC1 | Uncharacterized protein KIAA2030 | 2 | 20.53 | 2 |
| Q540A0 | Microtubule binding protein hook3 | 2 | 20.38 | 3 |
| Q5HZI1 | Angiotensin-II type 2 receptor-interacting protein | 2 | 20.05 | 3 |
| Q8BRB7 | Histone acetyltransferase MYST4 | 2 | 20.01 | 2 |
| Q8VD78 | Aldehyde dehydrogenase 16 family, member A1 | 2 | 19.93 | 5 |
| Q3TAP7 | Cerebellar degeneration-related 2 | 2 | 18.54 | 3 |

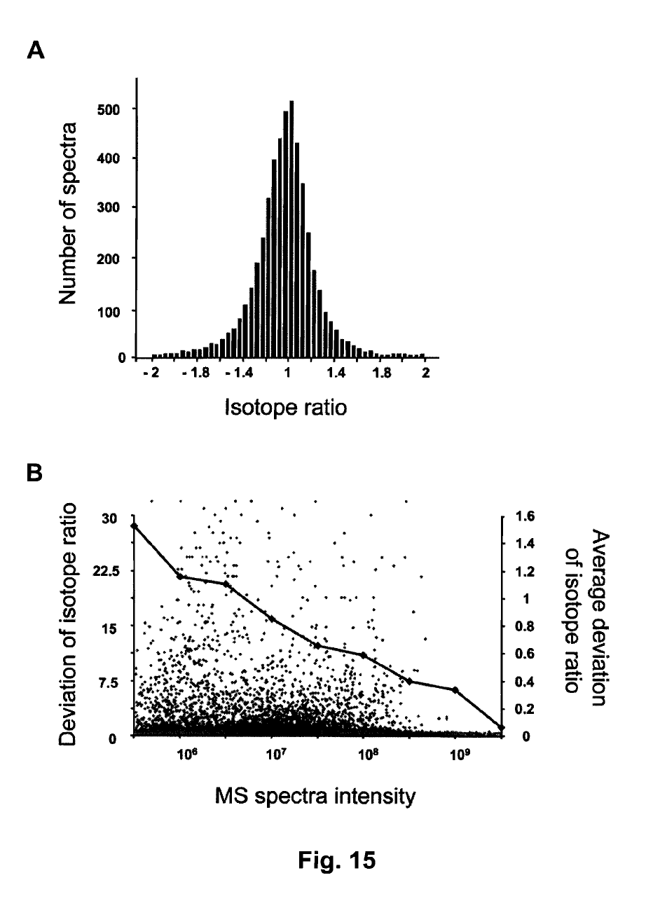

Variability of the isotope ratios was estimated for the subset of peptides that did not contain serine threonine and tyrosine residues and therefore were not expected to be phosphorylated. For those peptides, the ratios of intensities for the light- and heavy-labeled forms was visualized to assess the quality of the data (FIG. 15A). It was found that for all biological replicas average isotope ratio between the BIAP-treated and non-treated samples was close to 1 (0.95) with average standard deviation 0.26. The deviation of isotope ratio from 1 decreased with increase in intensity (FIG. 15B). Using the above distribution as the “null hypothesis” for each isotope ratio, a p value was calculated, representing the likelihood that a value equal to or higher than the given ratio can be obtained by chance. Peptides with statistically significant changes in isotope ratios in two of three replicas ratios were further validated by manual analysis of the spectra and extracted ion chromatograms.

For 7 unique peptides, significant increases of abundance in the samples treated with alkaline phosphatase was detected, suggesting that they may contain phosphorylated amino acid residues (Table V). All of them showed increased abundance only in the basal or only in the stimulated lipolytic state, suggesting that they are phosphorylated or dephosphorylated in response to the treatment of mice with the beta-3-adrenergic agonist.

TABLE V
Peptides with changed isotope ratios detected in LD purified from WAT of
control mice and those treated with beta-3 adrenergic agonist, CL316,243.

| | Mean isotope | | UniProt | |
|---|---|---|---|---|
| | ratio (SD) | | accession | |
| Peptide Sequence | Control | Treated | number | Protein name |
| THVDSLR | 1.47 | −1.12 | Q00623 | Apolipoprotein A-I |
| (SEQ ID NO: 43) | (0.16) | (0.2) | | |
| SHDTTSNTLTQLLAK | 1.88 | 1.11 | Q91VJ2 | Protein kinase C delta |
| (SEQ ID NO: 44) | (0.15) | (0.12) | | binding protein |
| FVEGLPINDFSR(E) | 2.1 | 3.08 | P08249 | Malate dehydrogenase, |
| (SEQ ID NO: 45) | (0.15) | (0.37) | | cytoplasmic |
| SVSEAALAQPEGLLGTDTLK | 1.6 | 5.1 | P54310 | Lipase, hormone |
| (SEQ ID NO: 46) | (0.28) | (0.45) | | sensitive |
| KLLVSSEDYGR | 1 | 3.47 | P16546 | Alpha II spectrin |
| (SEQ ID NO: 47) | (0.16) | (0.4) | | |
| LSLMEPESEFRDIDNPSAEAER | −1.06 | 1.63 | Q8CGN5 | Perilipin |
| (SEQ ID NO: 48) | (0.02) | (0.15) | | |
| GLSAPSCPNGLDDK | 1 | 1.4 | Q8CGN5 | Perilipin |
| (SEQ ID NO: 49) | (0.12) | (0.2) | | |

Data show mean values of 3 independent experiments.

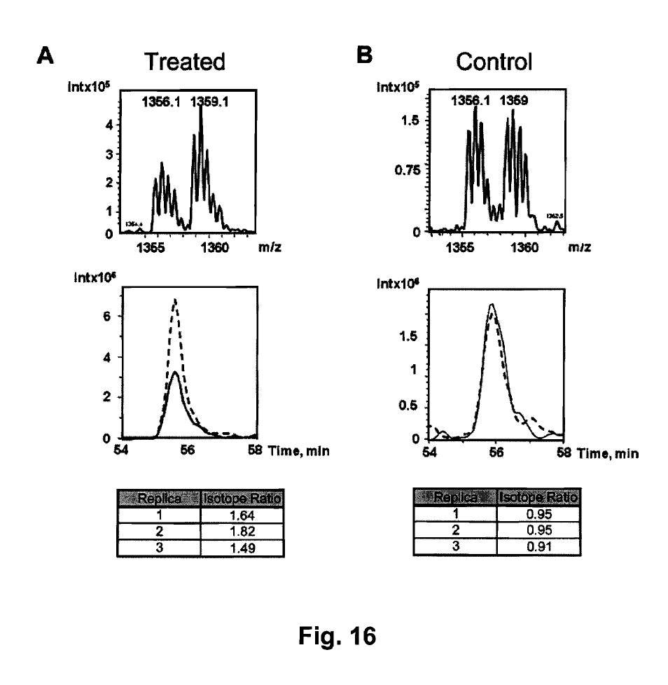

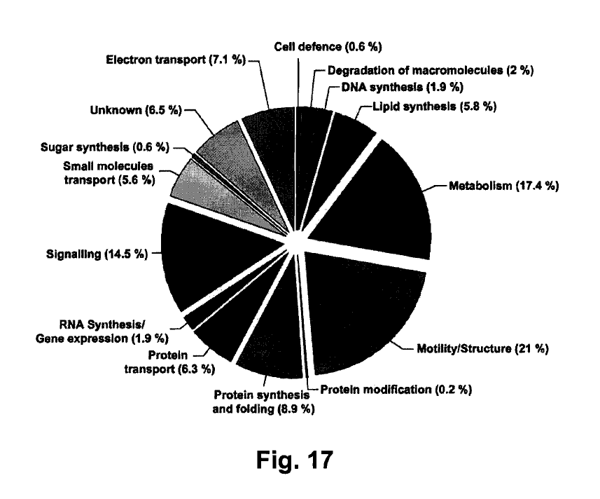

One of the most abundant peptides with the significantly changed isotope ratio was ⁴⁰⁹LSLMEPESEFRDIDNPSAEAER⁴³⁰ (SEQ ID NO: 48) from mouse perilipin A (FIG. 16A). This peptide was detected with charges +2 and +3 and both forms showed isotope ratio 1.6 in the samples from hormone-treated mice. In the samples from control mice, isotope ratios for the above peptides did not significantly differ from 1.0 suggesting the absence of phosphorylation (FIG. 16B). For another PLIN peptide, ⁴⁵⁸GLSAPSCPGLDDK⁴⁷⁰° (SEQ ID NO: 49), the isotope ratio was 1 in the control samples versus 1.4 in samples from treated animals. In similar fashion, a significant increase of the isotope ratio for the peptide ⁵⁵⁷SVSEAALAQPEGLLGTDTLKK⁵⁷⁷ (SEQ ID NO: 46) from HSL was detected, containing two previously-described phosphorylation sites, Ser-557 and Ser-559 (Ser-552 and Ser-554 in human HSL). Upon 133-adrenoceptor agonist treatment, the mean isotope ratio increased from 1.6 to 5.1, suggesting that ˜80% of this peptide in the LD of stimulated adipocytes is phosphorylated. In addition we detected potentially induced or suppressed phosphorylations in proteins involved in supporting the cell structure and motility (e.g., α-II spectrin), metabolism (e.g., apolipoprotein A1, malate dehydrogenase), and signalling (e.g., protein kinase C delta-binding protein) (FIG. 17).

In order to confirm the phosphorylation of LD peptides predicted on the basis of isotope ratios and to identify the phosphorylation sites, phosphopeptides enriched by IMAC chromatography using imidodiacetic acid Sepharose™ with immobilized Al³⁺ ions were analyzed. It was first confirmed that phosphopeptides are retained on this resin and can be specifically eluted with 50 mM sodium phosphate. Two control samples and two samples from stimulated mice were analyzed; each time the purification of phosphopeptides was performed using tryptic digest of 100 μg of LD proteins. The purified peptides were analyzed by LC-MS/MS using QTrap™ instrument and 1100 Series LC MSD SL instrument. Seventy percent of peptides enriched on imidodiacetic acid-Al³⁺ Sepharose™ were phosphorylated according to the MS/MS data. In total, 18 non-redundant phosphopeptides representing 13 phosphoproteins were identified (Table VI). Majority of these proteins were also identified by the global 2D-LC-MS/MS analysis of the LDS proteome.

TABLE VI
Phosphopeptides detected in LD after enrichment on IMAC resin.

| UniProt | | | SEQ | | | | | |
|---|---|---|---|---|---|---|---|---|
| Accession | | | ID | | | | | Error, |
| number | Protein name | Peptide | No: | Phosphosite | Score | m/z | z | ppm |
| Q4V9U2 | Perilipin | RVsDSFFRPSVMEPILGR | 50 | S492s | 16.5 | 725.39 | 3 | 503.0 |
| | | LAsGGADLALGSIEK | 51 | S174s | 13.7 | 741.39 | 2 | 32.4 |
| | | RLsTQFTAANELACR | 52 | S81s | 10.2 | 909.49 | 2 | 75.0 |
| | | LsLMEPESEFR | 53 | S410s | 10.0 | 709.35 | 2 | 60.5 |
| | | NsISVPIASTSDK | 54 | S130s | 9.52 | 699.85 | 2 | 29.7 |
| | | GLsAPSCPGLDDK | 55 | S460s | 9.1 | 698.85 | 2 | 79.7 |
| | | RVsTLANTLSR | 56 | S222s | 8.1 | 649.38 | 2 | 69.6 |
| P54310 | Lipase, hormone | RssQGVLHMPLYTSPIVK | 57 | S651s/S650s | 13.7 | 698.50 | 3 | 202.6 |
| | sensitive | SVsEAALAQPEGLL- | 58 | S559s | 14.5 | 694.01 | 3 | −2.6 |
| | | GTDTLKK | | | | | | |
| A2AUE1 | ER-associated | SLstsGESLYHVLGLDK | 59 | S10s/T11t/ | 12.5 | 629.37 | 3 | 106.7 |
| | dnaJ protein 3 | | | S12s | | | | |
| Q9Z1W4 | Growth/differentia- | LKPLTGEGtAGGGGGG- | 60 | T206t | 16.1 | 532.32 | 4 | −392.5 |
| | tion factor 11 | RRHIR | | | | | | |
| Q8JZM7 | Parafibromin | RsTIKTDLDDDITALK | 61 | S195s | 12.5 | 628.99 | 3 | 12.7 |
| Q8BRB8 | AHNAK | LRsEDGVEGDLGETQSR | 62 | S136s | 14.6 | 643.66 | 3 | 584.1 |
| Q8BIS8 | Alpha-1,6- | ALAEESRSTADVDsGAs- | 63 | S72s/S75s | 12.9 | 688.40 | 4 | −236.6 |
| | mannosyl- | MAGYADLKR | | | | | | |
| | glycoprotein beta- | | | | | | | |
| | N- | | | | | | | |
| | acetylglucosaminyl- | | | | | | | |
| | tras-ferase | | | | | | | |
| Q60VW7 | AT-hook- | AILKDsGTPSCPHCHPIR | 64 | S1208s | 14.6 | 532.07 | 4 | −333.5 |
| | containing | | | | | | | |
| | transcription | | | | | | | |
| | factor | | | | | | | |
| Q6ZQJ5 | DNA2-like | VLLSKDYTLIVGMPGtGK | 65 | T653t | 13.6 | 657.40 | 3 | −931.5 |
| | helicase | | | | | | | |
| Q4V8H8 | EH-domain | GPDEAIEDGEEGsEDDA- | 66 | S438s | 15.6 | 1278.95 | 2 | −47.3 |
| | containing protein 2 | EWVVTK | | | | | | |
| Q3U434 | Calcium | GNVVPsPLPTR | 67 | S42s | 12.4 | 608.82 | 2 | 19.9 |
| | regulated heat | | | | | | | |
| | stable protein 1 | | | | | | | |
| Q3TAF3 | Caspase 4 | HLsLRYGANFDIIGMK | 68 | S146s | 14.0 | 638.40 | 3 | −910.8 |
| A2AKJ2 | Vimentin | TYsLGSALRPSTSR | 69 | S39s | 18.0 | 525.97 | 3 | 90.6 |

Detected phosphorylated residues are shown in lower case. Proteins extracted from lipid droplets were digested; phosphopeptides were concentrated on IMAC-AI³⁺ column and analyzed in duplicate runs on LC-MS/MS.

Previously-reported phosphopeptides from PLIN and HSL were identified by our technique: peptide ⁴⁰⁹LSLMEPESEFR⁴¹⁸ (SEQ ID NO: 53) in PLIN, containing phospho-Ser-410, peptide ⁴⁵⁸GLSAPSCPGLDDK⁴⁷⁰ (SEQ ID NO: 55) in PLIN (phospho-Ser460) and HSL peptide ⁵⁵⁷SVSEAALAQPEGLLGTDTLKK⁵⁷⁷ (SEQ ID NO: 58) with phospho-Ser-559. Both sites in PLIN were recognized among the most probable PKA sites by the empirical computer algorithms (NetPhos 2.0 score values 0.81, 0.73 and 0.85, respectively; Blom et al., 1999. Journal of Molecular Biology 294(5):1351-1362). Furthermore, 5 other phosphorylation sites were identified in PLIN: Ser-81, Ser-130, Ser-174, Ser-222, and Ser-492, all in samples from stimulated mice. Isotope ratios for the peptides containing Ser-81, Ser-130, and Ser-174 measured in multiple replicas from control or treated animals did not statistically differ from 1 (Table VII), suggesting that PLIN isoforms phosphorylated at these residues may exist, but do not represent any significant fraction of total PLIN present in adipocytes. The peptide containing Ser-492, although detected, was always present in unlabeled state; in this peptide, the bulky Val residue is predicted to reduce the efficiency of labelling at the N-terminus (Fedjaev et al., 2007, supra). The peptide containing Ser-223 was never detected during the analysis of total digests. The HSL peptide containing phospho-Ser-650/Ser-651 was also identified, which was not detected during the analysis of total protein digests.

TABLE VII
Peptides detected for proteins with difference in isotopic ratios between the
basal and lipolytic conditions.

| | | SEQ ID | N- | | | Ratio | | Stdev | |
|---|---|---|---|---|---|---|---|---|---|
| score | Peptide | NO: | term | z | m/z | Control | Treated | Control | Treated |

A3KGU5 Alpha-II spectrin

| | | | | | | | | | |
|---|---|---|---|---|---|---|---|---|---|
| 21.43 | SADESGQALLAASHYASDEVR | 70 | F | 2 | 1177 | 1.1(3) | 1.14(3) | 0.16 | 0.28 |
| 20.82 | EAALTNEEVGADLEQVEVLQK | 71 | F | 3 | 821.3 | n/c(0) | n/c(0) | — | — |
| 20.42 | SQLLGSAHEVQR | 72 | F | 2 | 750.4 | 0.98(4) | 1.05(4) | 0.11 | 0.06 |
| 19.26 | MQHNLEQQIQAR | 73 | F | 2 | 835.9 | 1.09(2) | 0.94(3) | 0.1 | 0.12 |
| 19.02 | HQLLEADISAHEDR | 74 | F | 3 | 603.7 | n/c(0) | n/c(0) | — | — |
| 18.3 | SADESGQALLAASHYASDEVR | 75 | F | 3 | 785 | 1.41(2) | 1.1(3) | 1.02 | 0.41 |
| 18.1 | NQALNTDNYGHDLASVQALQR | 76 | H | 3 | 777 | n/a(0) | n/a(0) | — | — |
| 17.9 | HQAFEAELHANADR | 77 | F | 2 | 892.5 | 1(3) | 0.84(3) | 0.64 | 0.3 |
| 17.33 | HQAFEAELSANQSR | 78 | F | 2 | 881.9 | 0.85(3) | 1.24(2) | 0.26 | 0.33 |
| 17.14 | KFEEFQTDLAAHEER | 79 | H | 3 | 617.5 | n/a(0) | n/a(0) | — | — |
| 17.05 | EANELQQWITEK | 80 | F | 2 | 832.4 | 1.06(2) | 1.1(3) | 0.13 | 0.11 |
| 17.01 | LIQEQHPEEELIK | 81 | F | 2 | 891.5 | 1.06(3) | 1.09(4) | 0.26 | 0.14 |
| 16.56 | SADESGQALLAASHYASDEVR | 82 | H | 2 | 1089 | n/a(0) | n/a(0) | — | — |
| 16.5 | NQALNTDNYGHDLASVQALQR | 83 | F | 3 | 835.4 | 0.54(1) | n/d(0) | — | — |
| 16.25 | SSLSSAQADFNQLAELDR | 84 | F | 2 | 1064 | 1.18(3) | 0.99(3) | 0.36 | 0.41 |
| 16.08 | EAFLNTEDKGDSLDSVEALIK | 85 | F | 3 | 823.9 | n/c(0) | n/c(0) | — | — |
| 15.86 | LSDDNTIGQEEIQQR | 86 | F | 2 | 961.3 | 0.94(1) | 1.25(1) | — | — |
| 15.72 | DLTNVQNLQK | 87 | F | 2 | 674.3 | n/d(0) | 0.98(1) | — | — |
| 14.84 | NQALNTDNYGHDLASVQALQR | 88 | F | 2 | 1253 | 0.87(2) | 0.91(3) | 0.04 | 0.4 |
| 14.64 | GNAMVEEGHFAAEDVK | 89 | F | 2 | 940 | 1.12(2) | 1.1(2) | 0.28 | 0.09 |
| 14.1 | EAFLNTEDKGDSLDSVEALIK | 90 | H | 3 | 766 | n/a(0) | n/a(0) | — | — |
| 13.79 | AQLADSFHLQQFFR | 91 | F | 2 | 945 | n/d(0) | 1.04(1) | — | — |
| 13.37 | DLSSVQTLLTK | 92 | F | 2 | 690.3 | 1.08(2) | n/d(0) | 0.06 | — |
| 12.97 | NQALNTDNYGHDLASVQALQR | 93 | H | 2 | 1165 | n/a(0) | n/a(0) | — | — |
| 12.95 | TATDEAYKDPSNLQGK | 94 | F | 2 | 957.5 | 1.06(2) | n/d(0) | 0.06 | — |
| 12.67 | HQAFEAELHANADR | 95 | F | 3 | 597.6 | n/d(0) | 0.81(1) | — | — |
| 12.42 | HQLLEADISAHEDR | 96 | F | 2 | 908.1 | 0.96(1) | 1.04(2) | — | 0.08 |
| 12.39 | TKQDEVNAAWQR | 97 | F | 2 | 813.4 | 0.85(2) | 0.84(1) | 0.33 | — |
| 11.78 | EAFLNTEDKGDSLDSVEALIK | 98 | H | 2 | 1148 | n/a(0) | n/a(0) | — | — |
| 11.74 | DTEQVDNWMSK | 99 | F | 2 | 764.7 | n/d(0) | 1.03(1) | — | — |
| 11.72 | QQVAPMDDETGKELVLALYDYQEK | 100 | F | 3 | 989.3 | n/c(0) | n/c(0) | — | — |
| 11.46 | DLAALGDKVNSLGETAQR | 101 | F | 2 | 1020 | n/d(0) | 1.29(1) | — | — |
| 11.27 | RQDLEDSLQAQQYFADANEAESWMR | 102 | H | 3 | 1001 | n/a(0) | n/a(0) | — | — |
| [symbol] | [symbol] | [symbol] | [symbol] | [symbol] | [symbol] | [symbol] | [symbol] | — | — |
| 10.89 | LGESQTLQQFSR | 104 | F | 2 | 788.5 | 0.7(1) | n/d(0) | — | — |
| 10.77 | SSEEIESAFR | 105 | F | 2 | 668.3 | n/d(0) | 0.64(2) | — | 0.35 |
| 10.71 | ASAFNSWFENAEEDLTDPVR | 106 | H | 2 | 1150 | n/a(0) | n/a(0) | — | — |
| 10.08 | REELITNWEQIR | 107 | H | 2 | 793.9 | n/a(0) | n/a(0) | — | — |
| 9.74 | QGQIDNQYQSLLELGEK | 108 | H | 2 | 982.1 | n/a(0) | n/a(0) | — | — |
| 9.01 | EAIVTSEELGQDLEHVEVLQK | 109 | H | 2 | 1184 | n/a(0) | n/a(0) | — | — |

Q8CGN5 Perilipin

| | | | | | | | | | |
|---|---|---|---|---|---|---|---|---|---|
| 22.95 | LASGGADLALGSIEK | 110 | F | 2 | 789 | 0.97(4) | 0.98(4) | 0.17 | 0.13 |
| 22.37 | VSDSFFRPSVMEPILGR | 111 | H | 3 | 646.6 | n/a(0) | n/a(0) | — | — |
| 21.71 | GPTLLDGDLPEQENVLQR | 112 | F | 2 | 1085 | 1.14(4) | 1.05(4) | 0.19 | 0.13 |
| 21.31 | GPTLLDGDLPEQENVLQR | 113 | H | 3 | 665.3 | n/a(0) | n/a(0) | — | — |
| 21.11 | ETAEYAANTR | 114 | F | 2 | 650.7 | 0.78(1) | 0.71(1) | — | — |
| 20.95 | VLQLPWSGTCECFQK | 115 | F | 2 | 1020 | 1.03(3) | 0.98(4) | 0.18 | 0.1 |
| 20.87 | LASGGADLALGSIEK | 116 | H | 2 | 701.3 | n/a(0) | n/a(0) | — | — |
| 20.78 | LSTQFTAANELACR | 117 | F | 2 | 878.9 | 1.01(2) | 0.9(1) | 0.13 | — |
| 20.43 | GVQGASNLAAWSMEPVVR | 118 | F | 2 | 1024 | 0.88(1) | 1(2) | — | 0.16 |
| 20.41 | GPTLLDGDLPEQENVLQR | 119 | F | 3 | 723.9 | 1.07(3) | 1.01(4) | 0.24 | 0.23 |
| 20.03 | GVTDNVVDTVVHYVPLPR | 120 | F | 2 | 1078 | 0.98(3) | 0.93(3) | 0.08 | 0.06 |
| 20 | GVQGASNLAAWSMEPVVR | 121 | F | 3 | 683 | n/d(0) | 1(2) | — | 0.14 |
| 19.64 | GVTDNVVDTVVHYVPLPR | 122 | F | 3 | 719.3 | 1.03(2) | 1(3) | 0.35 | 0.19 |
| [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] |
| 18.65 | VLQLPWSGTCECFQK | 125 | F | 3 | 680.8 | 0.9(2) | 1.17(2) | 0 | 0.25 |
| 18.5 | GLDHLEEK | 126 | F | 2 | 558.3 | 1.15(4) | 1.05(4) | 0.23 | 0.19 |
| 18.15 | EAHPLVASVCNAYEK | 127 | F | 2 | 932 | 1.02(4) | 1.06(4) | 0.15 | 0.11 |
| 17.87 | NSISVPIASTSDK | 128 | F | 2 | 747.3 | 1.15(4) | 1.09(4) | 0.2 | 0.22 |
| 17.71 | DIDNPSAEAER | 129 | H | 2 | 608.8 | n/a(0) | n/a(0) | — | — |
| 16.69 | LSTQFTAANELACR | 130 | H | 2 | 791.4 | n/a(0) | n/a(0) | — | — |
| 16.5 | GVTDNVVDTVVHYVPLPR | 131 | H | 2 | 990.5 | n/a(0) | n/a(0) | — | — |
| 16.3 | EVTALPNPR | 132 | F | 2 | 587.2 | 1.08(3) | 1.1(4) | 0.36 | 0.12 |
| 15.35 | VVEFLLPPDKESAPSSGR | 133 | F | 3 | 701.8 | 1.04(2) | 1.11(4) | 0.39 | 0.38 |
| 14.38 | IPALQYPPEK | 134 | F | 2 | 665.5 | 1.02(3) | 1.01(4) | 0.12 | 0.13 |
| 13.71 | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | — | — |
| 8.85 | LASGGADLALGSIEK | 137 | H | 1 | 1402 | n/a(0) | n/a(0) | — | — |

P54310 Lipase, hormone sensitive

| | | | | | | | | | |
|---|---|---|---|---|---|---|---|---|---|
| 20.42 | (R)AAFPEGFHPR(R) | 138 | F | 2 | 652.3 | 1.08(3) | 1.14(2) | 0.23 | 0.26 |
| 16.89 | (R)QATEFCVQR(I) | 139 | F | 2 | 657.2 | 0.83(1) | 1.01(3) | — | 0.16 |
| [symbol] | [symbol] | [symbol] | [symbol] | [symbol] | [symbol] | [symbol] | [symbol] | [symbol] | [symbol] |
| 16.27 | (K)HCDLLGSTGER(I) | 141 | F | 2 | 710.3 | 0.89(2) | 1(4) | 0.15 | 0.17 |
| [symbol] | [symbol] | [symbol] | [symbol] | [symbol] | [symbol] | [symbol] | [symbol] | [symbol] | [symbol] |
| 13.91 | (R)LILTPPAAPLN(—) | 143 | F | 2 | 647.8 | 1.27(2) | 0.78(1) | 0.16 | — |
| 12.87 | (R)LLSLPPEAFEMPLTSDPR(L) | 144 | F | 3 | 732.6 | n/c(0) | n/c(0) | — | — |
| 11.23 | (K)CVSAYSGTEAEDHFDSDQK(A) | 145 | F | 3 | 774.6 | 1.09(1) | 0.92(1) | — | — |
| 10.71 | (R)DLGQPVTLK(V) | 146 | F | 2 | 572.5 | 1.26(1) | n/d(0) | — | — |
| 10.25 | (K)CVSAYSGTEAEDHFDSDQK(A) | 147 | F | 2 | 1164 | 1(3) | 1.08(1) | 0.05 | — |
| 9.37 | (R)DLGQPVTLK(V) | 148 | F | 1 | 1146 | 0.94(1) | n/d(0) | — | — |

O08855 Apolipoprotein A-I

| | | | | | | | | | |
|---|---|---|---|---|---|---|---|---|---|
| 20.41 | (K)VQPYLDEFQK(K) | 149 | F | 2 | 721.2 | n/d(0) | 0.97(2) | — | 0.11 |
| 18.33 | (K)SNPTLNEYHTR(A) | 150 | F | 2 | 753.9 | 0.94(4) | 1(4) | 0.19 | 0.04 |
| 15.85 | (R)TQLAPHSEQMR(E) | 151 | F | 2 | 736.9 | 1.3(1) | 0.99(1) | — | — |
| [symbol] | [symbol] | [symbol] | [symbol] | [symbol] | [symbol] | [symbol] | [symbol] | — | [symbol] |

Q8C064 Protein kinase C delta binding protein

| | | | | | | | | | |
|---|---|---|---|---|---|---|---|---|---|
| 18.31 | (R)LEANHGLLVAR(G) | 153 | F | 2 | 684.4 | 1.23(1) | 0.94(2) | — | 0.13 |
| [symbol] | [symbol] | [symbol] | [symbol] | [symbol] | [symbol] | [symbol] | [symbol] | [symbol] | [symbol] |
| 17.66 | (R)SHDTTSNTLTQLLAK(A) | 155 | H | 2 | 815.4 | n/a(0) | n/a(0) | — | — |
| 9.35 | (K)EDPEKPVLQIESAA(—) | 156 | F | 2 | 854.9 | n/d(0) | 0.74(1) | — | — |

P14152 Malate dehydrogenase, cytoplasmic

| | | | | | | | | | |
|---|---|---|---|---|---|---|---|---|---|
| 16.64 | (K)GEFITTVQQR(G) | 157 | F | 2 | 677.3 | 1.11(3) | 1.18(2) | 0.16 | 0.16 |
| [symbol] | [symbol] | [symbol] | [symbol] | [symbol] | [symbol] | [symbol] | [symbol] | [symbol] | — |
| 9.53 | (K)ELTEEKETAFEFLSSA(—) | 159 | H | 2 | 916.1 | n/a(0) | n/a(0) | — | — |

Proteins extracted from lipid droplets were digested, divided into two equal aliquots which were further modified with the light and heavy forms of SIMPL isotope tags. One part was dephosphorylated with Bovine intestinal alkaline phosphatase (BIAP). Then parts were pooled together, fractionated by 2D-HPLC and analysed by MS. L indicates that the peptide N-terminus was labeled with isotopic tag, U indicates non-modified peptide. Presumed phosphorylated peptides are shown in bold italics. Figures in brackets show numbers of replicates in which isotopic ratio was determined. For some peptides isotopic ratio was not available “n/a” (if the peptide was not labeled), non-detectable “n/d” (if one of the isotopic forms was not detected) or could not be calculated “n/c” (if the charge of the peptide was 3⁺).

Although the present invention has been described hereinabove by way of specific embodiments thereof, it can be modified, without departing from the spirit and nature of the subject invention as defined in the appended claims.

1. An affinity matrix comprising a solid support and a metal ion covalently attached thereto.

2. The affinity matrix of claim 1, wherein said metal ion has a 2⁺ or 3⁺ oxidation state.

3-4. (canceled)

5. The affinity matrix of claim 2, wherein said metal ion is aluminum, (ii) gallium, or (iii) a combination of (i) and (ii).

6. The affinity matrix of claim 1, wherein said solid support is a cross-linked polysaccharide.

7. The affinity matrix of claim 6, wherein said polysaccharide is dextran.

8-9. (canceled)

10. The affinity matrix of claim 6, wherein said polysaccharide comprises a monomeric moiety of formula I:

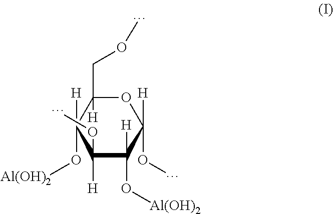

11. A method for preparing an affinity matrix comprising a solid support and a metal ion covalently attached thereto, said method comprising:
(a) contacting a solid support comprising hydroxyl groups attached thereto with an organometallic compound soluble in a first organic solvent; and
(b) contacting the solid support of (a) with a source of free hydroxyl groups,
thereby obtaining an affinity matrix comprising a solid support and a metal ion covalently attached thereto.

12. The method of claim 11, wherein said first organic solvent is a non-polar solvent.

13. The method of claim 11, wherein said organometallic compound is an alkylate of a metal.

14-17. (canceled)

18. The method of claim 13, wherein said alkylate of a metal is a trialkyl aluminum, a trialkyl gallium, or a combination thereof.

19. The method of claim 18, wherein said trialkyl aluminum is triethylalkyl aluminum (Al₂(Et)₆).

20. (canceled)

21. The method of claim 11, wherein said first organic solvent is hexane.

22. The method of claim 11, wherein said source of free hydroxyl groups is water.

23. The method of claim 11, further comprising (c) washing said solid support with a second organic solvent.

24. The method of claim 23, wherein said second organic solvent for washing said solid support is diethyl ether.

25. The method of claim 11, further comprising (d) drying the matrix.

26. (canceled)

27. The method of 11, wherein said solid support is a cross-linked polysaccharide matrix.

28-30. (canceled)

31. The method of claim 27, wherein said polysaccharide comprises a monomeric moiety of formula I:

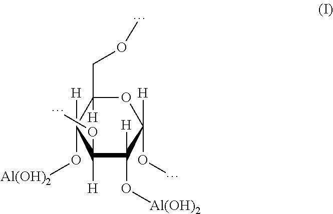

32. A method for obtaining a sample enriched in phosphorylated peptides from a protein-comprising sample, said method comprising:
(a) contacting the protein-comprising sample with matrix comprising a metal ion having a 3⁺ oxidation state;
(b) eluting the proteins from said matrix to obtain a phosphoprotein enriched sample;
(c) incubating the phosphoprotein enriched sample of (b) with a proteolytic enzyme thereby to obtain peptides;
(d) contacting the peptides of (c) with a matrix comprising a metal ion having a 3⁺ oxidation state; and
(e) eluting the peptides from the matrix of (d);
thereby obtaining a sample enriched in phosphorylated peptides.

33-36. (canceled)

37. The method of claim 32, wherein said matrix is an affinity matrix comprising a solid support and the metal ion covalently attached thereto.

38-94. (canceled)


SEQUENCE LISTING (159 sequences)

SEQ ID NO: 1 - protein, 14 aa, Bos primigenius
  MOD_RES (10)..(10): PHOSPHORYLATION
VPQLEIVPNSAEER

SEQ ID NO: 2 - protein, 16 aa, Bos primigenius
  MOD_RES (4)..(4): PHOSPHORYLATION
DIGSESTEDQAMEDIK

SEQ ID NO: 3 - protein, 16 aa, Bos primigenius
  MOD_RES (4)..(4): PHOSPHORYLATION
DIGSESTEDQAMEDIK

SEQ ID NO: 4 - protein, 20 aa, Gallus gallus
  MOD_RES (5)..(5): PHOSPHORYLATION
EVVGSAEAGVDAASVSEEFR

SEQ ID NO: 5 - protein, 12 aa, Bos primigenius
  MOD_RES (6)..(6): PHOSPHORYLATION
TVDMESTEVFTK

SEQ ID NO: 6 - protein, 12 aa, Bos primigenius
  MOD_RES (7)..(7): PHOSPHORYLATION
TVDMESTEVFTK

SEQ ID NO: 7 - protein, 17 aa, Bos primigenius
  MOD_RES (7)..(7): PHOSPHORYLATION
NMAINPSKENLCSTFCK

SEQ ID NO: 8 - protein, 16 aa, Bos primigenius
  MOD_RES (12)..(12): PHOSPHORYLATION
YKVPQLEIVPNSAEER

SEQ ID NO: 9 - protein, 17 aa, Homo sapiens
  MOD_RES (10)..(10): PHOSPHORYLATION
EALVEPASESPRPALAR

SEQ ID NO: 10 - protein, 17 aa, Homo sapiens
  MOD_RES (3)..(3): PHOSPHORYLATION
STSPAPADVAPAQEDLR

SEQ ID NO: 11 - protein, 20 aa, Homo sapiens
  MOD_RES (3)..(3): PHOSPHORYLATION
SATPPPAEPASLPQEPPKPR

SEQ ID NO: 12 - protein, 14 aa, Homo sapiens
  MOD_RES (11)..(11): PHOSPHORYLATION
LLNASAYRLQSECR

SEQ ID NO: 13 - protein, 14 aa, Homo sapiens
  MOD_RES (3)..(3): PHOSPHORYLATION
TASLTSAASIDGSR

SEQ ID NO: 14 - protein, 24 aa, Homo sapiens
  MOD_RES (11)..(11): PHOSPHORYLATION
LFQENSDIMNSLIESWCQNSEVKR

SEQ ID NO: 15 - protein, 17 aa, Homo sapiens
  MOD_RES (16)..(16): PHOSPHORYLATION
TINCSTQCIIKQIDHSK

SEQ ID NO: 16 - protein, 27 aa, Homo sapiens
  MOD_RES (8)..(8): PHOSPHORYLATION
LSAFRPWSPAVSASEKETSPHLPALIR

SEQ ID NO: 17 - protein, 20 aa, Homo sapiens
  MOD_RES (8)..(8): PHOSPHORYLATION
HSAEVAGYQDSIGQLESDLR

SEQ ID NO: 18 - protein, 26 aa, Homo sapiens
  MOD_RES (21)..(21): PHOSPHORYLATION
SAQGTGFELGQLQSIRSEGTTSTSYK

SEQ ID NO: 19 - protein, 17 aa, Homo sapiens
  MOD_RES (12)..(12): PHOSPHORYLATION
GQILVDFFNALSDATMK

SEQ ID NO: 20 - protein, 25 aa, Homo sapiens
  MOD_RES (6)..(6): PHOSPHORYLATION
GSRVVTVGSATHYVGTVDMADLHGR

SEQ ID NO: 21 - protein, 26 aa, Homo sapiens
  MOD_RES (9)..(9): PHOSPHORYLATION
AEEELAVDSVLEPEQEKMSQGFPSER

SEQ ID NO: 22 - protein, 18 aa, Homo sapiens
  MOD_RES (10)..(10): PHOSPHORYLATION
QMAEAQCKPYYSDYSRFR

SEQ ID NO: 23 - protein, 26 aa, Homo sapiens
  MOD_RES (25)..(25): PHOSPHORYLATION
GNLPTGTAGGVMEAGELGVAVKEETR

SEQ ID NO: 24 - protein, 22 aa, Homo sapiens
  MOD_RES (14)..(14): PHOSPHORYLATION
QISEDVDGPDNRESPENWEIVR

SEQ ID NO: 25 - protein, 26 aa, Homo sapiens
  MOD_RES (17)..(17): PHOSPHORYLATION
MIEAIGNQYVVARPVYSTKAFGEEFK

SEQ ID NO: 26 - protein, 16 aa, Homo sapiens
  MOD_RES (2)..(2): PHOSPHORYLATION
NTQNILVQNLSGGQKR

SEQ ID NO: 27 - protein, 23 aa, Homo sapiens
  MOD_RES (2)..(2): PHOSPHORYLATION
DTAGDASESALLKCIELSCGSVR

SEQ ID NO: 28 - protein, 12 aa, Homo sapiens
  MOD_RES (10)..(10): PHOSPHORYLATION
KEMNIQQLTTYR

SEQ ID NO: 29 - protein, 11 aa, Homo sapiens
  MOD_RES (6)..(6): PHOSPHORYLATION
GNVVPSPLPTR

SEQ ID NO: 30 - protein, 18 aa, Homo sapiens
  MOD_RES (17)..(17): PHOSPHORYLATION
GGDAEDVYYKSMPNLGSR

SEQ ID NO: 31 - protein, 16 aa, Homo sapiens
  MOD_RES (15)..(15): PHOSPHORYLATION
VLLLEAGPKDLLMGSK

SEQ ID NO: 32 - protein, 21 aa, Homo sapiens
  MOD_RES (3)..(3): PHOSPHORYLATION
RVSVCAETFNPDEEEEDNDPR

SEQ ID NO: 33 - protein, 25 aa, Homo sapiens
  MOD_RES (19)..(19): PHOSPHORYLATION
EGVAPLLGPASPTRLGTQSPSTSVR

SEQ ID NO: 34 - protein, 17 aa, Homo sapiens
  MOD_RES (9)..(9): PHOSPHORYLATION
LEEIILQITTVEAIIAR

SEQ ID NO: 35 - protein, 15 aa, Homo sapiens
IGGHGAEYGAEALER

SEQ ID NO: 36 - protein, 15 aa, Homo sapiens
IEDLSQQAQLAAAEK

SEQ ID NO: 37 - protein, 16 aa, Homo sapiens
VDATEESDLAQQYGVR

SEQ ID NO: 38 - protein, 12 aa, Homo sapiens
NIEDVIAQGVGK

SEQ ID NO: 39 - protein, 16 aa, Homo sapiens
LGAAPEEESAYVAGER

SEQ ID NO: 40 - protein, 16 aa, Homo sapiens
ILDSVGIEADDDRLNK

SEQ ID NO: 41 - protein, 22 aa, Mus musculus
LSLMEPESEFRDIDNPSAEAER

SEQ ID NO: 42 - protein, 21 aa, Bos primigenius
  MOD_RES (6)..(6): PHOSPHORYLATION
QMEAESISSSEEIVPNSVEQK

SEQ ID NO: 43 - protein, 7 aa, Mus musculus
THVDSLR

SEQ ID NO: 44 - protein, 15 aa, Mus musculus
SHDTTSNTLTQLLAK

SEQ ID NO: 45 - protein, 12 aa, Mus musculus
FVEGLPINDFSR

SEQ ID NO: 46 - protein, 20 aa, Mus musculus
SVSEAALAQPEGLLGTDTLK

SEQ ID NO: 47 - protein, 11 aa, Mus musculus
KLLVSSEDYGR

SEQ ID NO: 48 - protein, 22 aa, Mus musculus
LSLMEPESEFRDIDNPSAEAER

SEQ ID NO: 49 - protein, 13 aa, Mus musculus
GLSAPSCPGLDDK

SEQ ID NO: 50 - protein, 18 aa, Mus musculus
  MOD_RES (3)..(3): PHOSPHORYLATION
RVSDSFFRPSVMEPILGR

SEQ ID NO: 51 - protein, 15 aa, Mus musculus
  MOD_RES (3)..(3): PHOSPHORYLATION
LASGGADLALGSIEK

SEQ ID NO: 52 - protein, 15 aa, Mus musculus
  MOD_RES (3)..(3): PHOSPHORYLATION
RLSTQFTAANELACR

SEQ ID NO: 53 - protein, 11 aa, Mus musculus
  MOD_RES (2)..(2): PHOSPHORYLATION
LSLMEPESEFR

SEQ ID NO: 54 - protein, 13 aa, Mus musculus
  MOD_RES (2)..(2): PHOSPHORYLATION
NSISVPIASTSDK

SEQ ID NO: 55 - protein, 13 aa, Mus musculus
  MOD_RES (3)..(3): PHOSPHORYLATION
GLSAPSCPGLDDK

SEQ ID NO: 56 - protein, 11 aa, Mus musculus
  MOD_RES (3)..(3): PHOSPHORYLATION
RVSTLANTLSR

SEQ ID NO: 57 - protein, 18 aa, Mus musculus
  MOD_RES (2)..(2): PHOSPHORYLATION
RSSQGVLHMPLYTSPIVK

SEQ ID NO: 58 - protein, 21 aa, Mus musculus
  MOD_RES (3)..(3): PHOSPHORYLATION
SVSEAALAQPEGLLGTDTLKK

SEQ ID NO: 59 - protein, 17 aa, Mus musculus
  MOD_RES (3)..(5): PHOSPHORYLATION
SLSTSGESLYHVLGLDK

SEQ ID NO: 60 - protein, 21 aa, Mus musculus
  MOD_RES (9)..(9): PHOSPHORYLATION
LKPLTGEGTAGGGGGGRRHIR

SEQ ID NO: 61 - protein, 16 aa, Mus musculus
  MOD_RES (2)..(2): PHOSPHORYLATION
RSTIKTDLDDDITALK

SEQ ID NO: 62 - protein, 17 aa, Mus musculus
  MOD_RES (3)..(3): PHOSPHORYLATION
LRSEDGVEGDLGETQSR

SEQ ID NO: 63 - protein, 26 aa, Mus musculus
  MOD_RES (14)..(14): PHOSPHORYLATION
ALAEESRSTADVDSGASMAGYADLKR

SEQ ID NO: 64 - protein, 18 aa, Mus musculus
  MOD_RES (6)..(6): PHOSPHORYLATION
AILKDSGTPSCPHCHPIR

SEQ ID NO: 65 - protein, 18 aa, Mus musculus
  MOD_RES (16)..(16): PHOSPHORYLATION
VLLSKDYTLIVGMPGTGK

SEQ ID NO: 66 - protein, 23 aa, Mus musculus
  MOD_RES (13)..(13): PHOSPHORYLATION
GPDEAIEDGEEGSEDDAEWVVTK

SEQ ID NO: 67 - protein, 11 aa, Mus musculus
  MOD_RES (6)..(6): PHOSPHORYLATION
GNVVPSPLPTR

SEQ ID NO: 68 - protein, 16 aa, Mus musculus
  MOD_RES (3)..(3): PHOSPHORYLATION
HLSLRYGANFDIIGMK

SEQ ID NO: 69 - protein, 14 aa, Mus musculus
  MOD_RES (3)..(3): PHOSPHORYLATION
TYSLGSALRPSTSR

SEQ ID NO: 70 - protein, 21 aa, Mus musculus
SADESGQALLAASHYASDEVR

SEQ ID NO: 71 - protein, 21 aa, Mus musculus
EAALTNEEVGADLEQVEVLQK

SEQ ID NO: 72 - protein, 12 aa, Mus musculus
SQLLGSAHEVQR

SEQ ID NO: 73 - protein, 12 aa, Mus musculus
MQHNLEQQIQAR

SEQ ID NO: 74 - protein, 14 aa, Mus musculus
HQLLEADISAHEDR

SEQ ID NO: 75 - protein, 21 aa, Mus musculus
SADESGQALLAASHYASDEVR

SEQ ID NO: 76 - protein, 21 aa, Mus musculus
NQALNTDNYGHDLASVQALQR

SEQ ID NO: 77 - protein, 14 aa, Mus musculus
HQAFEAELHANADR

SEQ ID NO: 78 - protein, 14 aa, Mus musculus
HQAFEAELSANQSR

SEQ ID NO: 79 - protein, 15 aa, Mus musculus
KFEEFQTDLAAHEER

SEQ ID NO: 80 - protein, 12 aa, Mus musculus
EANELQQWITEK

SEQ ID NO: 81 - protein, 13 aa, Mus musculus
LIQEQHPEEELIK

SEQ ID NO: 82 - protein, 21 aa, Mus musculus
SADESGQALLAASHYASDEVR

SEQ ID NO: 83 - protein, 21 aa, Mus musculus
NQALNTDNYGHDLASVQALQR

SEQ ID NO: 84 - protein, 18 aa, Mus musculus
SSLSSAQADFNQLAELDR

SEQ ID NO: 85 - protein, 21 aa, Mus musculus
EAFLNTEDKGDSLDSVEALIK

SEQ ID NO: 86 - protein, 15 aa, Mus musculus
LSDDNTIGQEEIQQR

SEQ ID NO: 87 - protein, 10 aa, Mus musculus
DLTNVQNLQK

SEQ ID NO: 88 - protein, 21 aa, Mus musculus
NQALNTDNYGHDLASVQALQR

SEQ ID NO: 89 - protein, 16 aa, Mus musculus
GNAMVEEGHFAAEDVK

SEQ ID NO: 90 - protein, 21 aa, Mus musculus
EAFLNTEDKGDSLDSVEALIK

SEQ ID NO: 91 - protein, 14 aa, Mus musculus
AQLADSFHLQQFFR

SEQ ID NO: 92 - protein, 11 aa, Mus musculus
DLSSVQTLLTK

SEQ ID NO: 93 - protein, 21 aa, Mus musculus
NQALNTDNYGHDLASVQALQR

SEQ ID NO: 94 - protein, 16 aa, Mus musculus
TATDEAYKDPSNLQGK

SEQ ID NO: 95 - protein, 14 aa, Mus musculus
HQAFEAELHANADR

SEQ ID NO: 96 - protein, 14 aa, Mus musculus
HQLLEADISAHEDR

SEQ ID NO: 97 - protein, 12 aa, Mus musculus
TKQDEVNAAWQR

SEQ ID NO: 98 - protein, 21 aa, Mus musculus
EAFLNTEDKGDSLDSVEALIK

SEQ ID NO: 99 - protein, 11 aa, Mus musculus
DTEQVDNWMSK

SEQ ID NO: 100 - protein, 24 aa, Mus musculus
QQVAPMDDETGKELVLALYDYQEK

SEQ ID NO: 101 - protein, 18 aa, Mus musculus
DLAALGDKVNSLGETAQR

SEQ ID NO: 102 - protein, 25 aa, Mus musculus
RQDLEDSLQAQQYFADANEAESWMR

SEQ ID NO: 103 - protein, 11 aa, Mus musculus
KLLVSSEDYGR

SEQ ID NO: 104 - protein, 12 aa, Mus musculus
LGESQTLQQFSR

SEQ ID NO: 105 - protein, 10 aa, Mus musculus
SSEEIESAFR

SEQ ID NO: 106 - protein, 20 aa, Mus musculus
ASAFNSWFENAEEDLTDPVR

SEQ ID NO: 107 - protein, 12 aa, Mus musculus
REELITNWEQIR

SEQ ID NO: 108 - protein, 17 aa, Mus musculus
QGQIDNQYQSLLELGEK

SEQ ID NO: 109 - protein, 21 aa, Mus musculus
EAIVTSEELGQDLEHVEVLQK

SEQ ID NO: 110 - protein, 15 aa, Mus musculus
LASGGADLALGSIEK

SEQ ID NO: 111 - protein, 17 aa, Mus musculus
VSDSFFRPSVMEPILGR

SEQ ID NO: 112 - protein, 18 aa, Mus musculus
GPTLLDGDLPEQENVLQR

SEQ ID NO: 113 - protein, 18 aa, Mus musculus
GPTLLDGDLPEQENVLQR

SEQ ID NO: 114 - protein, 10 aa, Mus musculus
ETAEYAANTR

SEQ ID NO: 115 - protein, 16 aa, Mus musculus
VLQLPVVSGTCECFQK

SEQ ID NO: 116 - protein, 15 aa, Mus musculus
LASGGADLALGSIEK

SEQ ID NO: 117 - protein, 14 aa, Mus musculus
LSTQFTAANELACR

SEQ ID NO: 118 - protein, 18 aa, Mus musculus
GVQGASNLAAWSMEPVVR

SEQ ID NO: 119 - protein, 18 aa, Mus musculus
GPTLLDGDLPEQENVLQR

SEQ ID NO: 120 - protein, 18 aa, Mus musculus
GVTDNVVDTVVHYVPLPR

SEQ ID NO: 121 - protein, 18 aa, Mus musculus
GVQGASNLAAWSMEPVVR

SEQ ID NO: 122 - protein, 18 aa, Mus musculus
GVTDNVVDTVVHYVPLPR

SEQ ID NO: 123 - protein, 13 aa, Mus musculus
GLSAPSCPGLDDK

SEQ ID NO: 124 - protein, 22 aa, Mus musculus
LSLMEPESEFRDIDNPSAEAER

SEQ ID NO: 125 - protein, 16 aa, Mus musculus
VLQLPVVSGTCECFQK

SEQ ID NO: 126 - protein, 8 aa, Mus musculus
GLDHLEEK

SEQ ID NO: 127 - protein, 15 aa, Mus musculus
EAHPLVASVCNAYEK

SEQ ID NO: 128 - protein, 13 aa, Mus musculus
NSISVPIASTSDK

SEQ ID NO: 129 - protein, 11 aa, Mus musculus
DIDNPSAEAER

SEQ ID NO: 130 - protein, 14 aa, Mus musculus
LSTQFTAANELACR

SEQ ID NO: 131 - protein, 18 aa, Mus musculus
GVTDNVVDTVVHYVPLPR

SEQ ID NO: 132 - protein, 9 aa, Mus musculus
EVTALPNPR

SEQ ID NO: 133 - protein, 18 aa, Mus musculus
VVEFLLPPDKESAPSSGR

SEQ ID NO: 134 - protein, 10 aa, Mus musculus
IPALQYPPEK

SEQ ID NO: 135 - protein, 22 aa, Mus musculus
LSLMEPESEFRDIDNPSAEAER

SEQ ID NO: 136 - protein, 11 aa, Mus musculus
LSLMEPESEFR

SEQ ID NO: 137 - protein, 15 aa, Mus musculus
LASGGADLALGSIEK

SEQ ID NO: 138 - protein, 10 aa, Mus musculus
AAFPEGFHPR

SEQ ID NO: 139 - protein, 9 aa, Mus musculus
QATEFCVQR

SEQ ID NO: 140 - protein, 20 aa, Mus musculus
SVSEAALAQPEGLLGTDTLK

SEQ ID NO: 141 - protein, 11 aa, Mus musculus
HCDLLGSTGER

SEQ ID NO: 142 - protein, 21 aa, Mus musculus
SVSEAALAQPEGLLGTDTLKK

SEQ ID NO: 143 - protein, 11 aa, Mus musculus
LILTPPAAPLN

SEQ ID NO: 144 - protein, 18 aa, Mus musculus
LLSLPPEAFEMPLTSDPR

SEQ ID NO: 145 - protein, 19 aa, Mus musculus
CVSAYSGTEAEDHFDSDQK

SEQ ID NO: 146 - protein, 9 aa, Mus musculus
DLGQPVTLK

SEQ ID NO: 147 - protein, 19 aa, Mus musculus
CVSAYSGTEAEDHFDSDQK

SEQ ID NO: 148 - protein, 9 aa, Mus musculus
DLGQPVTLK

SEQ ID NO: 149 - protein, 10 aa, Mus musculus
VQPYLDEFQK

SEQ ID NO: 150 - protein, 11 aa, Mus musculus
SNPTLNEYHTR

SEQ ID NO: 151 - protein, 11 aa, Mus musculus
TQLAPHSEQMR

SEQ ID NO: 152 - protein, 7 aa, Mus musculus
THVDSLR

SEQ ID NO: 153 - protein, 11 aa, Mus musculus
LEANHGLLVAR

SEQ ID NO: 154 - protein, 15 aa, Mus musculus
SHDTTSNTLTQLLAK

SEQ ID NO: 155 - protein, 15 aa, Mus musculus
SHDTTSNTLTQLLAK

SEQ ID NO: 156 - protein, 14 aa, Mus musculus
EDPEKPVLQIESAA

SEQ ID NO: 157 - protein, 10 aa, Mus musculus
GEFITTVQQR

SEQ ID NO: 158 - protein, 12 aa, Mus musculus
FVEGLPINDFSR

SEQ ID NO: 159 - protein, 16 aa, Mus musculus
ELTEEKETAFEFLSSA